(19) United States
(12) Patent Application Publication
(10) Pub. No.: US 2025/0329423 A1
(43) Pub. Date: Oct. 23, 2025

(54) RELATING CROSS-FRACTIONATION CHARACTERIZATIONS TO POLYMER PROPERTIES

(71) Applicant: ExxonMobil Technology and Engineering Company, Spring, TX (US)

(72) Inventors: Michael A. LEAF, Baytown, TX (US); Hsu CHIANG, Houston, TX (US); Jay L. REIMERS, Houston, TX (US); Liezhong GONG, Basking Ridge, NJ (US); Donald A. WINESETT, Houston, TX (US); Kevin A. STEVENS, Raritan, NJ (US); Wen LI, Houston, TX (US); Scott R. HORTON, Annandale, NJ (US); Michael A. RISCH, Seabrook, TX (US); Fred D. EHRMAN, Houston, TX (US); Timothy D SHAFFER, Plainfield, NJ (US)

(21) Appl. No.: 19/061,655

(22) Filed: Feb. 24, 2025

Related U.S. Application Data
(60) Provisional application No. 63/636,448, filed on Apr. 19, 2024.

(51) Int. Cl. G16C 60/00; C08F 10/02; G16C 20/70

(52) U.S. Cl. CPC G16C 60/00; C08F 10/02; G16C 20/70

(57) ABSTRACT

A method for producing a polymer includes generating polymer properties of the polymer using a model that includes an algorithm with an input of a cross-fractionation characterization (CFC) of the polymer. The CFC is generated based on a user input regarding one or more portions of the CFC. The method also includes producing the polymer having the polymer properties.

### CROSS-REFERENCE TO RELATED APPLICATION

This application claims the benefit of U.S. Provisional Application No. 63/636,448, filed Apr. 19, 2024, entitled “RELATING CROSS-FRACTIONATION CHARACTERIZATIONS TO POLYMER PROPERTIES”, the entirety of which is incorporated by reference herein.

### BACKGROUND

The present disclosure relates generally to techniques for forming polymer, and, more specifically, to producing polyolefin polymer.

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Blown film techniques are common ways polyethylene films are manufactured. Such films can be used to make bags, plastic wrap, agricultural film, laminating films, barrier films, industrial packaging, shrink-wrap films, etc. Each application requires different film properties. The film properties depend on, among other things, the polyethylene composition and the extrusion conditions. The combination of compositions and conditions are significant. To determine the right compositions and conditions for the desired film properties, manufacturers rely on their experience and expertise to guide them through trial and error experimentation. This process to achieve the desired film properties is time consuming (e.g., the experience could take months and the expertise is developed over decades of film conversion) and is costly. More effective techniques are needed to at least narrow the combinations of compositions and conditions to be tested to produce useful and marketable films.

### BRIEF DESCRIPTION

In one embodiment, a method for producing a polymer includes generating polymer properties of the polymer using a model that includes an algorithm with an input of a cross-fractionation characterization (CFC) of the polymer. The CFC is generated based on a user input regarding one or more portions of the CFC. The method also includes producing the polymer having the polymer properties.

In another embodiment, a method for producing a polymer includes generating polymer film properties of a polymer film using a model comprising an algorithm with an input of a cross-fractionation characterization (CFC) of the polymer. The polymer film includes the polymer, and the CFC is generated based on a user input regarding one or more portions of the CFC. The method also includes producing the polymer.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings. The subject matter disclosed is capable of considerable modifications, alterations, combinations, and equivalents in form and function, as will occur to those skilled in the art and having the benefit of this disclosure.

FIG. 1 is a flow diagram of a process for producing a polymer, in accordance with one or more embodiments of the present disclosure;

FIG. 2A is a parity plot of predicted density vs. measured density for polymers in which the predicted densities are predicted using a machine learning-derived model, in accordance with one or more embodiments of the present disclosure;

FIG. 2B is another parity plot of predicted density vs. measured density for polymers in which the predicted densities are predicted using a Gaussian process model, in accordance with one or more embodiments of the present disclosure;

FIG. 3A is a parity plot of a measured dart impact strength vs. predicted dart impact strength for polymer films in which the predicted dart impact strengths are predicted using a machine learning-derived model, in accordance with one or more embodiments of the present disclosure;

FIG. 3B is another parity plot of a measured dart impact strength vs. predicted dart impact strength for polymer films in which the predicted dart impact strengths are predicted using a Gaussian process model, in accordance with one or more embodiments of the present disclosure;

FIG. 4 is a flow diagram of another process for producing a polymer, in accordance with one or more embodiments of the present disclosure;

FIG. 5 is a cross fractionation chromatography plot of polyethylene molecular weight vs. comonomer mole percent, in accordance with one or more embodiments of the present disclosure;

FIG. 6 is a plot of number of methylene units vs. number of comonomer units in polyethylene, in accordance with one or more embodiments of the present disclosure;

FIG. 7 is a graph of component density vs. molecular weight, in accordance with one or more current embodiments;

FIG. 8 is a graph plotting predicted melt index vs. molecular weight of backbones of polyethylenes with varying amounts of branching, in accordance with one or more current embodiments,

FIG. 9 is a graph plotting parity of predicted density vs. actual density of polymer resins, in accordance with one or more current embodiments;

FIG. 10 is a graph plotting parity of predicted melt index vs. actual melt index of polymer resins, in accordance with one or more current embodiments;

FIG. 11 is a graph plotting parity of predicted melt index ratio vs. actual melt index of polymer resins, in accordance with one or more current embodiments;

FIG. 12 is a flow diagram of yet another process for producing a polymer, in accordance with one or more current embodiments; and

FIG. 13 is a graphical user interface, in accordance with one or more current embodiments.

### DETAILED DESCRIPTION

One or more specific embodiments of the present disclosure will be described below. These described embodiments are only examples of the presently disclosed techniques. Additionally, in an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but may nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Before the present compounds, components, compositions, devices, software, hardware, equipment, configurations, schematics, systems, methods, and/or processes are disclosed and described, it is to be understood that unless otherwise indicated this invention is not limited to specific compounds, components, compositions, devices, software, hardware, equipment, configurations, schematics, systems, methods, or the like, as such may vary, unless otherwise specified. It is also to be understood that the terminology used herein is for the purpose of describing particular embodiments only and is not intended to be limiting.

Additionally, the methods and/or processes described herein can be performed on computing devices (or processor-based devices) that include a processor, a memory coupled to the processor, and instructions provided to the memory. The instructions are executable by the processor to perform the methods and/or processes described herein. The instructions can be a portion of code on a non-transitory computer readable medium. Any suitable processor-based device may be utilized for implementing all or a portion of embodiments of the present techniques, including without limitation personal computers, networks personal computers, laptop computers, computer workstations, mobile devices, multi-processor servers or workstations with (or without) shared memory, high performance computers, and the like. Moreover, embodiments may be implemented on application specific integrated circuits (ASICs) or very large scale integrated (VLSI) circuits.

The present disclosure relates to several techniques that may utilize one or more models regarding polymers, components of polymers (e.g., monomers or compounds utilized to synthesize polymers), and polymer films.

The techniques described herein relate to modeling regarding polymers, polymer films, and reactor parameters utilized to generate polymers. For example, as described below, a model relating reactor parameters to polymer properties and/or polymer film properties may be generated, reactor parameters may be generated for an input indicative of target polymer properties and/or target polymer film properties based on the model (or one or more algorithms thereof), and polymer may be produced using the generated reactor parameters. Polymer film may be made using the produced polymer, for example, by extruding the polymer (e.g., in blown extrusion, cast extrusion, or other known processes for making film from polymers).

As also described herein, polymer molecular ensembles may be modeled as sets of polymer components of the molecular ensemble. For example, a polymer resin may include one or more molecular ensembles. A model may characterize physical properties of the polymer components of molecular ensembles, and a model relating the physical properties of the polymer components to polymer properties and/or polymer film properties may also be generated. Polymer component parameters may be generated for an input indicative of target polymer properties and/or target polymer film properties based on the model(s) (or one or more algorithms thereof), and polymer (e.g., polymer component(s)) may be generated using the generated polymer component parameters. Polymer film may be made using the generated polymer, for example, by extruding the polymer.

### Definitions

As used herein, the term “metallocene catalyst” is defined to comprise at least one transition metal compound containing one or more substituted or unsubstituted cyclopentadienyl moiety (Cp) (typically two Cp moieties) in combination with a Group 4, 5, or 6 transition metal, such as (but not limited to), zirconium, hafnium, and titanium.

As used herein, the term “polyethylenes” (PEs) encompasses polyethylene homopolymers and copolymers of greater than or equal to 50 mol % ethylene-derived content and less than or equal to 50 mol % C3-C20 alpha-olefin-derived content. Examples include ethylene-butene, ethylene-hexene, and ethylene-octene polyethylene copolymers (wherein the C3-C20 α-olefin comonomer is, respectively, 1-butene, 1-hexene, and 1-hexene). “Metallocene polyethylenes” are polyethylenes that are synthesized using a catalyst system comprising a metallocene catalyst.

As used herein, the term “mixed catalyst” refers to two or more catalysts. For example, a mixed catalyst may be two or more different catalysts co-supported on the same carrier such as a bimodal catalyst. In a mixed catalyst, one or more of the different catalysts may be metallocene catalysts. For example, a mixed catalyst may include one metallocene catalyst, two metallocene catalysts, or three or more metallocene catalysts; and/or it can include at lease one metallocene and at lease one non-metallocene catalyst (such as a chromium catalyst, a Zeigler-Natta type catalyst, an iron catalyst, or other catalyst useful for polymerization of monomers, especially of ethylene and/or alpha-olefins). When metallocene catalysts are discussed herein, they may be activated as is well known in the art of metallocene catalysis; furthermore, catalysts discussed herein may optionally be supported, as is also well known.

As used herein, the term “mixed catalyst system” refers to a system that utilizes a mixed catalyst composition, and may also encompass other components utilized for the catalyst to effectively polymerize monomers; for example, it can include an optional support and/or activators (to yield the active form of the catalyst). A mixed catalyst system may be considered a dual catalyst system when the mixed catalyst only includes two catalysts.

As used herein, unless otherwise specified, melt index (MI), alternatively referred to as melt flow rate (MFR), is measured at 190° C. and 2.16 kg per ASTM D1238-13.

As used herein, unless otherwise specified, heavy load melt index (HLMI), which can also be referred to as heavy load melt flow rate (HLMFR), is measured at 190° C. and 21.6 kg per ASTM D1238-13.

As used herein, unless otherwise specified, flow rate ratio (FRR) is the HLMFR divided by the MFR.

As used herein, unless otherwise specified, a bulk density (φ is measured per ASTM D1505-10.

As used herein, a molecular weight can be reported as number average (Mn), weight average (Mw), or z-average (Mz) as determined by gel permeation chromatography (GPC) as described in “Modern Size-Exclusion Liquid Chromatography, Practice of Gel Permeation and Gel Filtration Chromatography” by W. W. Yau, J. J. Kirkland and D. D. Bly (John Wiley & Sons, 1979); further reference to this text will indicate the chapter and page of “GPC-Yau.”

As used herein, a polydispersity index (PDI) or molecular weight distribution (MWD) refers to Mw/Mn.

As used herein, the term “blown film extrusion” refers to a process where a polymer melt is extruded through a circular die followed by bubble-like expansion.

As used herein, the term “melt temperature” (MT) refers to the polymer melt temperature at the extruder die, which has units of ° F. unless otherwise specified.

As used herein, the term “output rate” (OR) is the extruder throughput, which has units of lb/hr unless otherwise specified.

As used herein, the term “process time” is the calculated time for the polymer melt to travel from the die exit to the frost line height (FLH), which has units of mm unless otherwise specified.

As used herein, the term “strain rate” (STR) is calculated according to EQ. 1, which has units of l/s unless otherwise specified:

$$STR = ( {V}_{film} - {V}_{die} ) / FLH \qquad EQ . 1$$

where V_{film} is the polymer film travel velocity above the frost line, and V_{die} is the polymer travel velocity at the extruder.

As used herein, the term “draw down ratio” (DDR) is calculated according to EQ. 2, which is unitless.

$$DDR = {V}_{film} / {V}_{die} \qquad EQ . 2$$

As used herein, the term “process time” (PT) is calculated according to EQ. 3, which is seconds(s) unless otherwise specified.

$$PT = FLH / [ {V}_{film} - {V}_{die} ) / log ( {V}_{film} / {V}_{die} ) ] \qquad EQ . 3$$

As used herein, the term “machine direction tear” (TearMD) refers to Elmendorf Tear, which is measured per ASTM D1922-15 but is reported as a normalized value relative to the film thickness with the units of grams per mil (g/mil), unless otherwise specified.

As used herein, the term “model” refers to a system of one or more algorithms.

As used herein, the term “algorithm” carries its normal meaning and refers without limitation to any series of repeatable steps that result in a discrete value or values. For example, an algorithm may include any mathematical, statistical, positional, or relational calculation between any numbers of user-specified, preset, automatically-determined, or industry- or system-acceptable data elements. In several embodiments, various algorithms may be performed on subject data elements in relation to a previously defined data evaluation sample in order to produce a single, meaningful data value.

As used herein, a “molecular ensemble” refers to a grouping or arrangement of molecules, including, but not limited to polymer molecules. For example, a molecular ensemble may include polymer molecules, including homopolymers and/or copolymers. Accordingly, a molecular ensemble of a polymer (e.g., a homopolymer, a copolymer, or a both a homopolymer and a copolymer) may be representative of how polymer molecules are arranged (e.g., in three-dimensional space and/or relative to one another).

The terms “non-transitory, computer-readable medium,” “tangible machine-readable medium,” or the like refer to any tangible storage that participates in providing instructions to a processor for execution. Such a medium may take many forms, including but not limited to, non-volatile media, and volatile media. Non-volatile media includes, for example, NVRAM, or magnetic or optical disks. Volatile media includes dynamic memory, such as main memory. Computer-readable media may include, for example, a floppy disk, a flexible disk, hard disk, magnetic tape, or any other magnetic medium, magneto-optical medium, a CD-ROM, any other optical medium, a RAM, a PROM, and EPROM, a FLASH-EPROM, a solid state medium like a holographic memory, a memory card, or any other memory chip or cartridge, or any other physical medium from which a computer can read. When the computer-readable media is configured as a database, it is to be understood that the database may be any type of database, such as relational, hierarchical, object-oriented, and/or the like. Accordingly, exemplary embodiments of the present techniques may be considered to include a tangible storage medium or tangible distribution medium and prior art-recognized equivalents and successor media, in which the software implementations embodying the present techniques are stored.

Setting Reactor Parameters Based on Polymer Properties and/or Polymer Film Properties

Techniques described herein relate to modeling regarding polymers, polymer films, and reactor parameters utilized to produce polymers. For instance, as described below, a model relating reactor parameters to polymer properties and/or polymer film properties may be generated, reactor parameters may be generated for an input indicative of target polymer properties and/or target polymer film properties based on the model (or one or more algorithms thereof), and polymer may be produced using the generated reactor parameters. Such production can include, e.g., polymerization of a plurality of monomers (which may be of one or more types, e.g., one or more of ethylene, propylene, butene, or any other C₂-C₂₀ olefin, preferably α-olefin, where ethylene is considered an α-olefin for purposes of this disclosure). Polymer film may be made using the produced polymer, for example, by extruding the polymer.

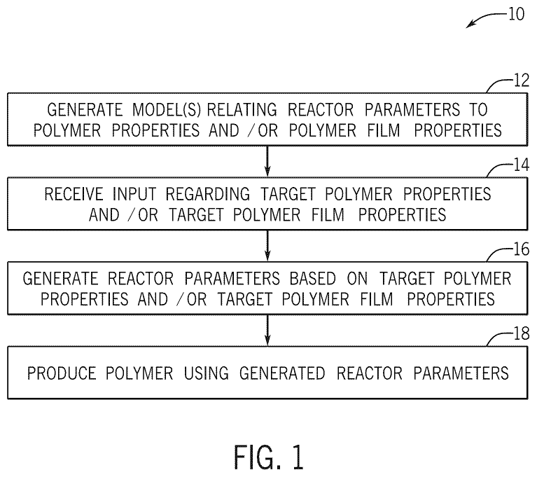

Bearing the foregoing in mind, FIG. 1 is a flow diagram of a process 10 for producing a polymer, such as polyethylene. The process 10 may be performed on one or more computing devices (or processor-based devices) that include a processor, a memory coupled to the processor, and instructions provided to the memory. The instructions are executable by the processor to perform the methods and/or processes described herein. The instructions can be a portion of code on a non-transitory computer readable medium. Any suitable processor-based device may be utilized for implementing all or a portion of embodiments of the present techniques, including without limitation personal computers, networks personal computers, laptop computers, computer workstations, mobile devices, multi-processor servers or workstations with (or without) shared memory, high performance computers, and the like. Moreover, embodiments may be implemented on application specific integrated circuits (ASICs) or very large scale integrated (VLSI) circuits. The process 10 generally includes generating one or more models relating reactor parameters to polymer properties and/or polymer film properties (process block 12), receiving an input regarding target polymer properties and/or target polymer film properties (process block 14), generating reactor parameters based on the target polymer properties and/or target polymer film properties (process block 16), and producing the polymer using the reactor parameters (process block 18).

At process block 12, one or more models relating reactor parameters to polymer properties and/or polymer film properties may be generated. The one or more models may utilize machine learning and may be particular to a particular polymer and/or polymerization method (and/or a reactor used for a particular polymerization method or technique). For example, a Gaussian process model (GPM) technique may be used to develop a model for a (mixed catalyst) gas phase reactor (e.g., a gas phase polyethylene (GPPE) reactor). More particularly, experimental data can be collected through design of experiment (DOE) via an active learning protocol or using classical screening-follow up-response surface experiment designs. The resulting polymers (e.g., polymer resins) may then be characterized for their polymer properties, which may include density (e.g., bulk density), melt index (MI), melt index ratio (MIR), or any combination thereof. The melt index, may be the melt flow rate (MFR) and may also be called a melt flow index. Melt index ratio may be the flow rate ratio (FRR).

A machine-learning technique (ElasticNet, LASSO, Ridge, Stepwise, etc.) and/or a GPM technique may then be used over the collected (GPPE) process dataset to develop the following quantitative functional relationships, as represented by EQ. 4 below, which is an example equation for a scenario according to some embodiments involving production of polyethylene, and in particular an ethylene-hexene polyethylene copolymer, using a polymerization catalyst in a condensed-mode (or super-condensed-mode) gas phase polymerization process utilizing one or more induced condensing agents (ICAs).

$$ρ / MI / MIR = f ( CatComp , T , H 2 / C 2 = Gas , C 6 / C 2 = Flow , iC 5 , C 2 = PP , t ) \qquad EQ . 4$$

### In EQ. 4:

ρ is density of the polymer (e.g., g/cm³), although it is noted that for this and other terms of EQ. 4, any term of EQ. 4 can be any acceptable units of measurement, so long as it is consistent with underlying data used in building (e.g., used in fitting) the predictive model.
CatComp is activated catalyst composition, meaning that the model is specific to a particular activated catalyst composition. For example, if activated Metallocene Catalyst A (having a specific chemical composition, activated using a particular activator, and, optionally, supported on a specific support) is used in generating the underlying data used to fit the model, then the model's predicted values (e.g., ρ, MI, and MIR) will be specific to a polymer (e.g., polyethylene) made using Metallocene Catalyst A. The CatComp term also allows flexibility for consideration of a single-catalyst or a multi-catalyst system. When a single catalyst is utilized, the catalyst composition (which may be the CatComp) may include the catalyst and an amount of the catalyst utilized. Furthermore, for catalyst compositions in which two or more catalysts are utilized (a multi-catalyst system), the catalyst composition (which may be the CatComp) may include the catalysts utilized as well as the amounts or relative amounts of the catalysts used (e.g., a ratio of the amounts of catalysts used).
T is reactor temperature (in the exemplified case of a gas phase polymerization process for polyethylene, reactor temperature T is taken as the temperature of the fluidized bed within the gas phase reactor). As noted above for density, T can be in any suitable unit of measurement (e.g., ° C., ° F.) so long as it is consistent with reactor temperature measured in connection with underlying data collection to which the model is fit.
H2/C2-Gas is the hydrogen to ethylene (H2/C2=) gas ratio in the polymerization reactor (molar or mass ratio of hydrogen to ethylene fed to the gas phase polymerization process; as long as one is consistent in using either molar or mass ratio between measured data and the model). More broadly, for polymerization of a different primary monomer besides ethylene, this term can be taken as the hydrogen to primary monomer ratio.
C6/C2=Flow is the hexene to ethylene (C6/C2=) feed flow ratio. This, similar to the H2/C2=Gas term, represents the particular instance where ethylene is the primary or predominant (>50 mol % units derived therefrom in the polymer produced) monomer in the polymerization, and hexene is a comonomer (<50% units derived therefrom in the polymer produced), for production of ethylene-hexene polyethylene copolymer as just noted, with similar note that either mass or molar ratio can be used as long as one is consistent. More broadly, this term could be taken as comonomer to monomer feed flow ratio.
iC5 is the isopentane composition in the reaction medium—and in particular, is taken as the ratio of iC5 flow rate to ethylene (primary monomer) flow rate. More broadly, it is the ratio of ICA (induced condensing agent) flow rate to primary monomer (e.g., ethylene) flow rate, for the scenario of gas phase polymerization in a condensed or super-condensed mode. Furthermore, if a different ICA (other than iC5) or mixture of ICAs is used, one can normalize this term based on the latent heat sink (or removal) capacity of the ICA or ICA mixture (that is, for an ICA having, say, 1.2× the heat sink capacity of iC5, this term can be multiplied by 1.2, recognizing this term is meant to capture the ICA's heat-sink effects on the reaction conditions being modeled).
C2=PP is the partial pressure of ethylene in reactor (e.g., ethylene monomer used for the exemplified production of polyethylene), and more broadly can be taken as partial pressure of the monomer (primary monomer) in the reactor.
t is the reactor residence time of the material within the reactor (for gas phase fluidized bed reactors, for example, this can be taken as bed weight divided by polymer granule production rate).

In other words, the models may define relationships between polymer properties (which, per this example, may include density (e.g., resin density), melt index (MI), and melt index ratio (MIR)) and reactor parameters, which per this example may include one or more of activated catalyst compositions (CatComp), reactor temperature (T), comonomer/monomer flow ratios (again, here, we exemplify hexene to ethylene (C6/C2=) flow ratio), hydrogen to monomer gas ratio (e.g., hydrogen-to-ethylene (H2/C2=) gas ratio), reactor residence time (t), monomer partial pressure (here, ethylene or C2=partial pressure (C2=PP)), ICA composition (here, isopentane (iC5) composition), or any combination thereof. That is, the use of “ρ/MI/MIR” is indicative of at least one of density, melt index, or melt index ratio being defined as a function of one or more reactor parameters (as opposed to signifying division). Furthermore, additional terms can be modeled as a function of the above-summarized reactor parameters; for instance, one or more of catalyst productivity and hydrogen consumption rate could be modeled in addition to (or instead of) the above-mentioned density, MI, and MIR; recognizing that these properties of the reaction can also be modeled as a function of the above-noted parameters.

As noted above, one or more models may be generated using machine learning techniques, which may include but are not limited to ElasticNet, LASSO, Ridge, Stepwise, and GPM. For GPM, the model may utilize a kernel function provided as EQ. 5 below.

$$K ( {x}_{i} , {x}_{j} ) = {σ}^{2} exp ( - {( {x}_{i} - {x}_{j} )}^{n} / 2 {L}^{2} ) + {δ}_{ij} {σ}_{noise}^{2} \qquad EQ . 5$$

where xᵢ is a vector of the prediction variables at experiment i, xⱼ is a vector of the prediction variables at experiment j, σ and L are model hyper-parameters obtained by fitting the model to experimental data, δᵢⱼ is equal to zero when i and j are different and equal to one when i and j are equivalent, σ²ₙₒᵢₛₑ is a positive value (e.g., integer or decimal value) greater than zero and less than or equal to one-hundred, and n is a value (e.g., integer or decimal value) ranging from zero to five, inclusive. σ²ₙₒᵢₛₑ may be utilized to ensure stability of the algorithm (e.g., when using GPM), and the value of σ²ₙₒᵢₛₑ will often be a value greater than zero and less than or equal to one. The value of n may alternatively range from one or three, inclusive. Additionally, the value of n may range from one and one-half (1.5) to two and one-half (2.5). For example, the value of n may be 1.5, 1.75, 2, 2.25, 2.5, or any other value in the range of 1.5 to 2.5.

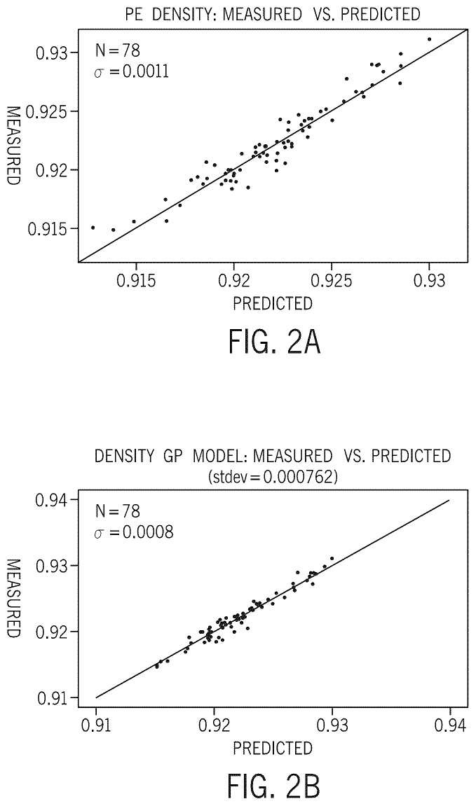

Continuing with the drawings, FIG. 2A and FIG. 2B are parity plots of predicted density vs. measured density of polymers (in these figures, the polymers are polyethylenes and in particular ethylene-hexene polyethylene copolymers). In particular, the plot of FIG. 2A is illustrative of results of a predictive model generated using (conventional) machine learning techniques such as ElasticNet, LASSO, Ridge, and/or Stepwise. FIG. 2B is illustrative of results of a predictive model in which a GPM was employed (e.g., a model using a GPM-derived algorithm to predict polymer densities). As shown in FIG. 2A, utilizing conventional machine learning techniques resulted in a standard deviation (σ) of 0.0011, whereas, as shown in FIG. 2B, utilizing GPM resulted in a standard deviation of 0.0008. For other polymer properties (e.g., melt index and melt index ratio), lower standard deviations were also achieved utilizing GPM-derived algorithms compared to algorithms derived utilizing other machine learning techniques. Accordingly, utilizing GPM may result in more accurate modeling of polymer properties relative to conventional machine learning techniques.

Returning to FIG. 1 and the discussion of the process 10, the model(s) may also relate the polymer properties (e.g., density, MI, MIR, or any combination thereof) to polymer film properties, which may be properties of a film made from the polymer (e.g., using a blown film extrusion process). More specifically, the polymer film properties may be properties present when using a particular set of fabrication conditions using blown film extrusion. For example, the particular set of fabrication conditions may include a gauge of 1 mil (i.e., one-thousandth of one inch), blow up ratio (BUR, which may be the ratio of bubble diameter to the die diameter) of 2.5, a die gap of 60 mil, and a die diameter of 6 inches. This is just an example; others utilizing these techniques will of course find it beneficial to define their own relevant set of parameters (e.g., a gauge of 0.5 mil, 10 mil, 15 mil, etc.; BUR of 3, 3.5, etc.; die gaps of 30, 35, 40, 45, 50, 55, 60, 65, 70, or 75 mil, etc.; and similarly varying die diameters, based upon the particular film equipment of interest to their modeling efforts). Efforts may also be made to control additional film fabrication parameters, including frost line height, melt temperature, and output rate as constant values. Accordingly, experimental data can be collected through design of experiment (DOE) via an active learning protocol or using classical screening-follow up-response surface experiment designs. The resulting polymers (e.g., polymer resins) may be subjected to blown film extrusion to make polymer films, and the films may then be characterized for their (film) properties (e.g., mechanical and optical properties), which may include secant modulus (e.g., one or both of machine direction secant modulus and transverse direction secant modulus), dart impact strength, haze total, tear (e.g., one or both of machine direction tear and transverse direction tear), tensile strength (e.g., one or both of machine direction tensile strength and transverse direction tensile strength), puncture break force, puncture break energy, and break elongation (e.g., in one or both the machine and transverse directions).

A machine-learning technique (ElasticNet, LASSO, Ridge, Stepwise . . . ) and/or a GPM technique may then be used over the collected (GPPE) process dataset to develop the following quantitative functional relationships, as represented by EQ. 6 below.

$$polymer film properties ( at fixed fabrication ) = f ( ρ , MI , MIR , FLH , {T}_{M} , OR ) \qquad EQ . 6$$

When utilizing GMP, the kernel function as described in EQ. 5 above may be utilized. Accordingly, in one example embodiment, the polymer film properties (e.g., secant modulus (e.g., one or both of machine direction secant modulus and transverse direction secant modulus), dart impact strength, haze total, tear (e.g., one or both of machine direction tear and transverse direction tear), tensile strength (e.g., one or both of machine direction tensile strength and transverse direction tensile strength), puncture break force, puncture break energy, break elongation (e.g., in one or both the machine and transverse directions), or any combination thereof) may be characterized as a function of both polymer properties (density of the polymer used to make the film (φ, melt index of the polymer (MI), melt index ratio of the polymer (MIR)) and film production conditions (temperature of the polymer melt in the film production equipment, e.g., in the extruder (T_{M}); frost line height of the film production equipment (FLH); and output rate of the film production equipment (OR)). It is noted that, as with other models discussed herein, measurement units of each term can be any suitable measurement units, as long as one is consistent between the model and the underlying data (e.g., if underlying data to build model includes temperature, e.g., T_{M}, in ° C., then the model term T should be in ° C.). In other embodiments, another combination of density, melt index, melt index ratio, temperature of polymer melt, frost line height, and output rate (e.g., fewer than each of these properties) may be utilized. As such, the one or more models may relate polymer film properties to polymer properties (e.g., at user-defined fabrication settings), along with basic film production parameters such as frost line height and output rate. As noted, one could further limit the model to predicting the desired polymer properties (right side of EQ. 6) needed to achieve desired polymer film properties (left side of EQ. 6) using a fixed frost line height and output rate, thereby further simplifying EQ. 6 (allowing one to ‘delete’ the FLH and OR terms), leaving only polymer properties on the right side of EQ. 6.

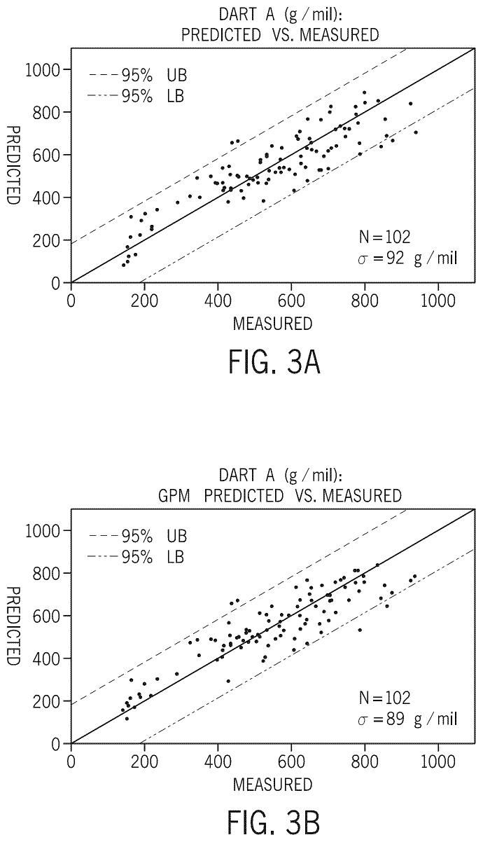

FIG. 3A and FIG. 3B are parity plots of measured dart impact strength vs. predicted dart impact strength for polymer (in this example, polyethylene copolymers made using a mixed catalyst system, and having a range of different density, MI, and MIR) films at particular user-defined fabrication settings (in this case, using defined fabrication parameters of 1 mil, BUR of 2.5, die gap of 60 mil, and die diameter of 6 in). Furthermore, FLH and OR were held constant across the experiments (approx. 20 in. FLH and 188 lbs/hr OR), as was temperature of the polymer melt, so that predicted film properties as a function of polymer properties were obtained and able to be compared against the measured film properties achieved from using polymers having the input polymer properties. In particular, FIG. 3A is illustrative of results of a predictive model generated using (conventional) machine learning techniques such as ElasticNet, LASSO, Ridge, and/or Stepwise. FIG. 3B is illustrative of results of a predictive model in which a GPM was employed on the same data (e.g., a model using a GPM-derived algorithm to predict polymer densities). As shown in FIG. 3A, utilizing conventional machine learning techniques resulted in a standard deviation (σ) of 92 g/mil in the predicted vs measured Dart A values of the polymer films, whereas, as shown in FIG. 3B, utilizing GPM resulted in a standard deviation of 89 g/mil in the predicted vs measured Dart A values of the polymer films. For other reactor parameters, lower standard deviations were also achieved utilizing GPM-derived algorithms compared to algorithms derived utilizing other machine learning techniques. Accordingly, utilizing GPM may result in more accurate modeling of polymer properties relative to conventional machine learning techniques.

Returning to FIG. 1 and the discussion of the process 10, at process block 14, an input regarding target polymer properties and/or target polymer film properties may be received. For example, a computing device implementing the algorithms or models of the present disclosure may receive a user input indicative of target polymer properties of a polymer to be generated and/or target polymer film properties of a polymer film to be generated. The target polymer properties may include density, melt index, melt index ratio, or any combination thereof. The target polymer film properties may include secant modulus (e.g., one or both of machine direction secant modulus and transverse direction secant modulus), dart impact strength (e.g., a value in g/mil), haze total, tear (e.g., one or both of machine direction tear and transverse direction tear), tensile strength (e.g., one or both of machine direction tensile strength and transverse direction tensile strength), puncture break force, puncture break energy, break elongation (e.g., in one or both the machine and transverse directions), or any combination thereof.

At process block 16, reactor parameters (e.g., use of a mixed catalyst system) may be generated based on the target polymer properties and/or target polymer film properties. For example, one or more of the models may be utilized to determine reactor parameters (e.g., of a GPPE reactor) to produce a polymer (e.g., polyethylene) having polymer properties that are the target polymer properties. Indeed, in the case of the user input at process block 14 including density, melt index, and melt index ratio, a model in accordance with EQ. 4 may be used, and such a model may also utilize EQ. 5 (e.g., in the case of using GMP). When the user input includes target polymer film properties, a polymer (or polymer properties) resulting in the target polymer film properties may be determined utilizing the models described herein, and reactor settings may be determined based on the polymer (properties). For example, as described above, the polymer film properties may be defined as a function (or functions) of polymer properties (as exemplified, density, melt index, and melt index ratio), and optionally also of film production parameters (temperature of polymer melt, frost line height, and output rate), provided that the function can be simplified by targeting constant temperature of melt, frost line height, and/or output rate. Preferably, all three of the film production parameters are held constant, leaving the function (EQ. 6) to be further simplified such that desired (target) polymer film properties are expressed as a function of density, melt index, and melt index ratio. And, because reactor parameters may, in turn, be defined as a function of polymer density, melt index, and melt index ratio, (e.g., as shown in EQ. 4), then (target) polymer properties determined based on one or more target polymer film properties may accordingly be utilized (with a model) to determine reactor parameters to be utilized to produce a polymer that will result in a polymer film (e.g., after being subjected to blown film extrusion) having the target polymer film properties indicated by the user input received at process block 14.

As discussed above, the reactor parameters (e.g., for a GPPE reactor) may include activated catalyst compositions (CatComp), reactor temperature (T) (fluidized bed temperature in a gas phase fluidized bed polymerization reactor), comonomer to monomer (e.g., hexene to ethylene (C6/C2=)) flow ratio, hydrogen to monomer (e.g., hydrogen to ethylene (H2/C2=)) gas ratio, reactor residence time (t), C2=partial pressure (C2=PP), isopentane (iC5) composition, or any combination thereof, wherein each of these reactor parameters was described in detail (both in the present exemplary form and for general application) above in connection with EQ. 4. In addition, other reactor parameters are contemplated; for example, CatComp (based on activated catalyst composition) could readily be used to back-calculate a catalyst feed rate based upon observed measurements of activation rate for the given catalyst composition, if desired for simplicity of output to determine a desired feed rate of a given catalyst with a given activator. As such, the reactor parameters determined at process block 16 may include any of the foregoing reactor parameters just discussed. Examples of mixed catalysts or mixed catalyst systems that may be included in the reactor parameters may include those described in U.S. Patent Application Publication No. US 2020/0071437, which is hereby incorporated by reference. The catalyst composition may include the catalyst(s) utilized, which may include at least one metallocene catalyst. When a single catalyst is utilized, the catalyst composition (which may be the CatComp) may include the catalyst and an amount of the catalyst utilized, as previously described. Furthermore, for catalyst composition in which two or more catalysts are utilized, the catalyst composition (which may be the CatComp) may include the catalysts utilized as well as the amounts or relative amounts of the catalysts used (e.g., a ratio of the amounts of catalysts used).

At process block 18, the polymer may be generated using the reactor parameters. For instance, the polymer may be polyethylene that may be generated in a GPPE reactor using the reactor parameters generated at process block 16. In this manner, a polymer having polymer properties that are equal or approximately equal (e.g., within 5%, within 2%, within 1%) to the target polymer properties (e.g., as specified in a user input received at process block 14) may be generated. Moreover, the polymer may be extruded (e.g., using blown film extrusion) to produce a polymer film having polymer film properties that are equal to or substantially equal to the target polymer film properties. To do so, the polymer may be heated to generate molten polymer, and the molten polymer may be extruded using the blown film extrusion properties discussed above.

Polymers, including polyethylene, may be made by any suitable polymerization method including solution polymerization, slurry polymerization, and gas phase polymerization (including condensed mode and super-condensed mode) using supported or unsupported catalyst systems, such as a system incorporating a metallocene catalyst.

As alluded to previously, metallocene catalysts generally require activation with a suitable co-catalyst, or activator, in order to yield an “active metallocene catalyst” (i.e., an organometallic complex with a vacant coordination site that can coordinate, insert, and polymerize olefins). Active catalyst systems generally include not only the metallocene complex, but also an activator, such as an alumoxane or a derivative thereof (preferably methyl alumoxane), an ionizing activator, a Lewis acid, or a combination thereof. Alkylalumoxanes (typically methyl alumoxane and modified methylalumoxanes) are particularly suitable as catalyst activators. The catalyst system may be supported on a carrier, typically an inorganic oxide or chloride or a resinous material such as, for example, polyethylene or silica.

Zirconium transition metal metallocene-type catalyst systems may be particularly suitable. Non-limiting examples of metallocene catalysts and catalyst systems useful in producing polymer (e.g., polyethylene) include those described in, U.S. Pat. Nos. 5,466,649, 6,476,171, 6,225,426, and 7,951,873, and in the references cited therein, all of which are fully incorporated herein by reference. Particularly useful catalyst systems include supported dimethylsilyl bis(tetrahydroindenyl) zirconium dichloride.

Supported polymerization catalyst may be deposited on, bonded to, contacted with, or incorporated within, adsorbed or absorbed in, or on, a support or carrier. In another embodiment, the metallocene is introduced onto a support by slurrying a presupported activator in oil, a hydrocarbon such as pentane, solvent, or non-solvent, then adding the metallocene as a solid while stirring. The metallocene may be finely divided solids. Although the metallocene is typically of very low solubility in the diluting medium, it is found to distribute onto the support and be active for polymerization. Very low solubilizing media such as mineral oil (e.g., KAYDO™ or DRAKOL™) or pentane may be used. The diluent can be filtered off and the remaining solid shows polymerization capability much as would be expected if the catalyst had been prepared by traditional methods such as contacting the catalyst with methylalumoxane in toluene, contacting with the support, followed by removal of the solvent. If the diluent is volatile, such as pentane, it may be removed under vacuum or by nitrogen purge to afford an active catalyst. The mixing time may be greater than 4 hours, but shorter times are suitable.

Typically in a gas phase polymerization process, a continuous cycle is employed where in one part of the cycle of a reactor, a cycling gas stream, otherwise known as a recycle stream or fluidizing medium, is heated in the reactor by the heat of polymerization. This heat is removed in another part of the cycle by a cooling system external to the reactor. (See e.g., U.S. Pat. Nos. 4,543,399, 4,588,790, 5,028,670, 5,317,036, 5,352,749, 5,405,922, 5,436,304, 5,453,471, 5,462,999, 5,616,661, and 5,668,228.) To obtain the first additional polyethylene polymers, individual flow rates of ethylene, comonomer, and hydrogen may be controlled and adjusted to obtain the desired polymer properties.

Accordingly, the presently disclosed techniques enable reactor parameters to be determined for producing polymers and/or polymer films with particular properties. Additionally, because properties of polymers and polymer films can be predicted (reactor parameters may be determined without the polymers and/or polymer films to be made (e.g., by testing varying reactor parameters), time and resources that would otherwise be used to test reactor settings, polymer properties, and polymer film properties.

In accordance with present embodiments, a method for producing a polymer includes generating reactor parameters using a model that includes a Gaussian process model-derived algorithm with an input of target polymer properties of the polymer. The reactor parameters include a catalyst composition having at least one metallocene catalyst. Additionally, the method includes producing the polymer using the reactor parameters.

In the method, the polymer may include a polyethylene. Producing the polymer may include producing the polyethylene using a gas phase polyethylene reactor operating using the reactor parameters. The catalyst composition may include a mixed catalyst composition that includes two or more metallocene catalysts. The reactor parameters may include a reactor bed temperature, a hexene to ethylene flow ratio, a hydrogen to ethylene gas ratio, a reactor residence time, a partial pressure of ethylene, an isopentane composition, or any combination thereof. In the method, the reactor parameters may include the reactor bed temperature, the hexene to ethylene flow ratio, the hydrogen to ethylene gas ratio, the reactor residence time, the partial pressure of ethylene, and the isopentane composition.

In the method, the target polymer properties may include a bulk density, a melt flow rate, a flow rate ratio, or any combination thereof (wherein each is as described, for example, in connection with EQ. 4). The target polymer properties may include the bulk density, the melt flow rate, and the flow rate ratio.

In the method, the Gaussian process model-derived algorithm may include the kernel function of EQ. 5.

Various methods may include heating a polymer obtained according to the foregoing, to form a molten polymer and extruding the molten polymer using one or more blown film extrusion conditions to produce a film.

In accordance with the techniques described herein, a method for producing a polymer includes generating reactor parameters using a model comprising a Gaussian process model-derived algorithm with an input of target polymer film properties of a polymer film that includes the polymer. The reactor parameters include a catalyst composition having at least one metallocene catalyst. The method also includes producing the polymer using the reactor parameters.

In the method, the polymer may be or include a polyethylene. Producing the polymer may include using a gas phase polyethylene reactor operating using the reactor parameters. Additionally, the catalyst composition may include a mixed catalyst composition having two or more metallocene catalysts. Furthermore, the reactor parameters may include a reactor bed temperature, a hexene to ethylene flow ratio, a hydrogen to ethylene gas ratio, a reactor residence time, a partial pressure of ethylene, and an isopentane composition.

In the method, the target polymer film properties may include: a machine direction secant modulus, a transverse direction secant modulus, a dart impact strength, a haze total, a machine direction tear, a transverse direction tear, a machine direction tensile strength, a transverse direction tensile strength, a puncture break force, a puncture break energy, or any combination thereof, preferably 2 or more, 3 or more, 4 or more, or even all of the foregoing.

In the method, the algorithm may define the target polymer film properties as a function of a plurality of polymer properties. At least a portion of the plurality of the polymer properties as a function of the reactor parameters. In the method, the Gaussian process model-derived algorithm may include the kernel function of EQ. 5.

A computing device (e.g., for controlling a polymer production system or the production of a polymer) may include a processor, a memory coupled to the processor, and instructions provided to the memory. The instructions are executable by the processor to perform any processes or methods described herein.

### Relating Polymer Molecular Ensembles to Polymer Properties

The present disclosure also relates to modeling relating to polymer molecular ensembles, for instance, to synthesize polymers (e.g., polymer resins) and/or polymer films with desired properties. As discussed below, polymer molecular ensembles may be modeled as sets of polymer components of the molecular ensemble. For example, a polymer resin may include one or more molecular ensembles. A model may characterize physical properties of the polymer components of molecular ensembles, and a model relating the physical properties of the polymer components to polymer properties and/or polymer film properties may also be generated. Polymer component parameters may be generated for an input indicative of target polymer properties and/or target polymer film properties based on the model(s) (or one or more algorithms thereof), and polymer (e.g., polymer component(s)) may be produced using the generated polymer component parameters. Polymer film may be made using the generated polymer, for example, by extruding the polymer.

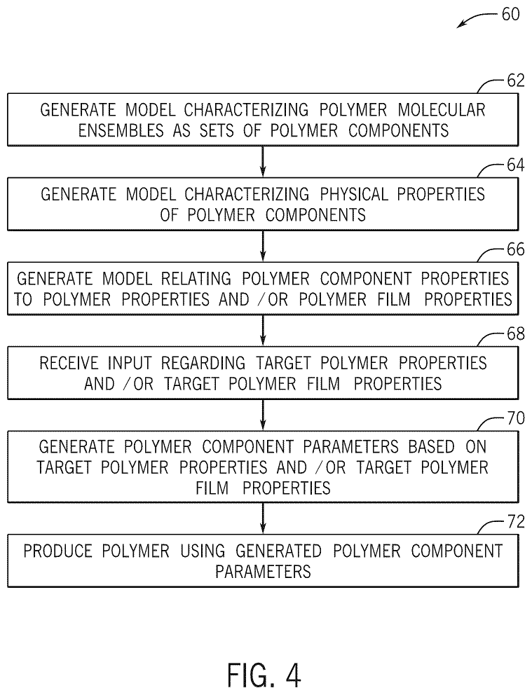

Bearing this in mind, FIG. 4 is a flow diagram of a process 60 for producing a polymer, such as polyethylene. The process 60 may be performed on one or more computing devices (or processor-based devices) that include a processor, a memory coupled to the processor, and instructions provided to the memory. The instructions are executable by the processor to perform the methods and/or processes described herein. The instructions can be a portion of code on a non-transitory computer readable medium. Any suitable processor-based device may be utilized for implementing all or a portion of embodiments of the present techniques, including without limitation personal computers, networks personal computers, laptop computers, computer workstations, mobile devices, multi-processor servers or workstations with (or without) shared memory, high performance computers, and the like. Moreover, embodiments may be implemented on application specific integrated circuits (ASICs) or very large scale integrated (VLSI) circuits. The process 60 generally includes generating a model characterizing polymer molecular ensembles as sets of polymer components (process block 62), generating a model characterizing physical properties of the polymer components (process block 64), generating a model relating polymer component properties to polymer properties and/or polymer film properties (process block 66), receiving an input regarding target polymer properties and/or target polymer film properties (process block 68), generating polymer components parameters based on the target polymer properties and/or the target polymer film properties (process block 70), and producing a polymer using the generated polymer component parameters (process block 72).

At process block 62, a model characterizing polymer molecular ensembles as sets of polymer components may be generated. Each polymer molecular ensemble may include polymer molecules (e.g., several polymer molecules of a polymer resin), and the model may describe mathematically the polymer ensembles based on the polymer components that make up the polymer ensembles. For example, a polymer resin may be characterized as one or more polymer ensembles that are representative of the polymer (e.g., homopolymer, copolymer, or homopolymer and copolymer) molecules in the polymer ensemble. Polymer (e.g., polyethylene) molecular ensembles can be characterized by the molecular weight (MW), MW distributions (MWD), short chain branch (SCB) amounts, short chain branch distribution (SCBD), long chain branch (LCB) amounts, and long chain branch distribution (LCBD). Long chain branches may be branches that are long enough to entangle and be rheologically significant, while short chain branches may be branches that do not entangle or are not rheologically significant. As an example, for polyethylene such as linear low-density polyethylene (LLDPE), examples of short chain branches may include branches incorporated via copolymerization of ethylene with one or more olefinic comonomers (e.g., 1-butene, 1-hexene, 1-octene). Each of these characteristics (e.g., MW, MWD, SCB, SCBD, LCB and LCBD) at the near molecular level can be quantified using a sample (e.g., a few grams) of a polymer resin polymerized from a lab scale reactor by advanced characterization techniques such as gas phase chromatography (GPC) with multiple detectors (e.g., GPC-3D or GPC-4D), cross fractionation chromatography, rheology, 1H-NMR, 13C-NMR, and light scattering. For example, to quantify long chain branches, GPC may be coupled with light scattering and/or viscometer detectors that calculate a coil dimension change vs. linear references, and the resulting signals can be used to calculate the ratio of solution coil size of linear versus branched (known as g′) as a function of molecular weight.

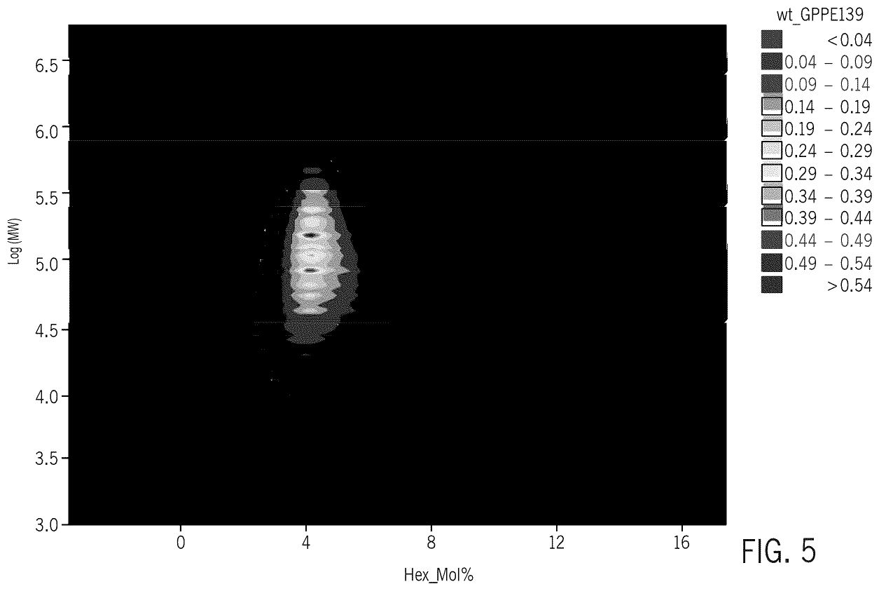

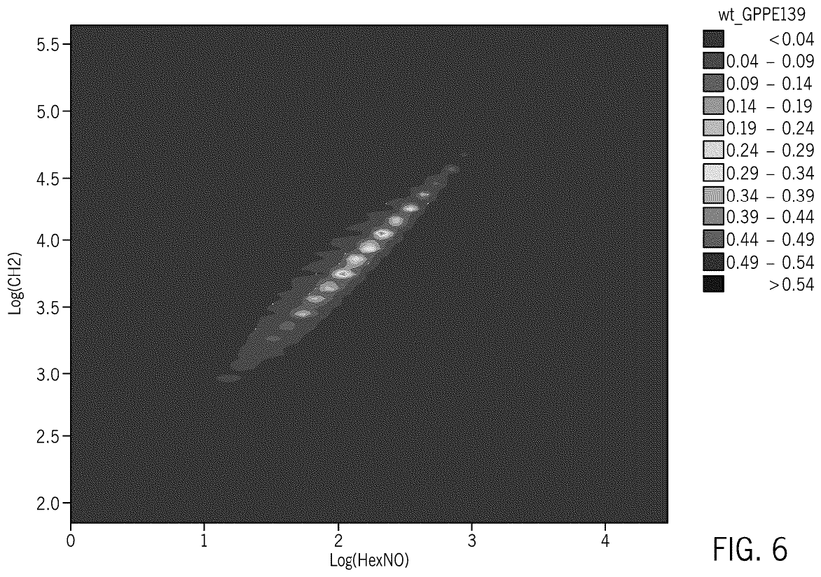

Polymer components of polymer ensembles may be characterized by number of methylene (CH₂) units in the polymer component as well as the number of hexene units (e.g., hexene-derived units) in the polymer component. The polymer may also be characterized by weight percent of the polymer (e.g., among the polymers of a polymer resin). Bearing this in mind, FIG. 5 is a cross fractionation chromatography (CFC) plot of polyethylene molecular weight (log MW) vs. comonomer (e.g., hexene) mole percent. The cross fractionation chromatography plot of FIG. 5 may be transformed into the graph of FIG. 6, which is a Log-scale plot of the number of methylene (CH2) units (R) vs. the number of hexene units (Br) in polyethylene, using “wt” to represent the abundance (e.g., weight percent) of a specific polyethylene species. Therefore, the following vector can be used to describe a polyethylene component in a polyethylene resin: R1000Br10 wt0.05, where “R1000” represents the polyethylene species has 1000 CH2 units, “Br10” indicates there are ten hexene units in the polyethylene species, and “wt0.05” defines the abundance of this polymer (polyethylene) component in the polyethylene resin as being 0.05 wt %. The values for R, Br and wt can be readily derived from the plot shown in FIG. 6.

The above vector can be extended to include LCB information, for example, when written as: R1000Br10 wt0.05LCB2, where “LCB2” represents that this polyethylene species has two long chain branches. The amount of long chain branches may be obtained from rheology test data. Additionally, to further enhance the fidelity and robustness of the model (e.g., of the components of a polymer), the molecular weight (MW) and comonomer content results from cross fractionation chromatography can be validated and reconciled with analytical data from, e.g., nuclear magnetic resonance imaging (NMR) for comonomer contents and light scattering for MW and MWD.

Accordingly, the model generated in process block 62 may mathematically describe polymer ensembles (e.g., of polymer resins) as sets of components (e.g., polymers) that make up the polymer ensembles. As discussed below, the model generated at process block 62 may be used in the development of a model defining a quantitative relationship between polyethylene molecular ensembles and polyethylene resins. In other words, polymer resins may be characterized by the polymer components characterized by the model generated at process block 62.

Referring to FIG. 4, at process block 64, a model characterizing physical properties of polymer components may be generated. In particular, the model may be generated using one or more functions (which may be included in the models(s) or one or more algorithms of the function(s)) to capture physical trends of polymers per polymer fundamental trends, and data can then be used according to the captured trends to estimate model parameters. For example, the polymer component physical properties may include a density of a polymer component, a melt index (also referred to as a melt flow rate) of a polymer component, or both the density and the melt index of the polymer component. The model generated at process block 64 may be included in the model generated at process block 62.

The polymer fundamental trends may include a first trend and a second trend. The first trend may be that comonomers reduce polymer component density. For example, a polyethylene component density decreases with the incorporation of more comonomer such as 1-butene, 1-hexene, or 1-octene (e.g., due to more branches from a backbone of the polymer component forming as more comonomer is incorporated). The second trend is that homo-polymer (e.g., homo-polyethylene) density decreases as molecular weight increases (e.g., without taking into consideration comonomers that may also be used). For example, a first polyethylene chain having seven carbon atoms would have a lower molecular weight (and be physically smaller) than a second polyethylene chain having fifteen carbon atoms. This would allow for the molecules of the first polyethylene chain to be more densely packed relative to molecules of the second polyethylene, thereby resulting in a higher density as molecular weight decreases (and, conversely, lower density of molecular weight increases).

Bearing this in mind, the density of a component i may be modeled as provided below in EQ. 7 (as well as EQ. 8, and EQ. 9, which define variables of EQ. 7):

$${ρ}_{i} = {ρ}_{min} + \frac{( {ρ}_{max} - {ρ}_{min} )}{1 + exp ( - k * ( {log}_{10} n {CH}_{2} ) )} \qquad EQ . 7$$

where ρᵢ is the density of the component i; ρₘᵢₙ is minimum density among all possible components for the polymer being modeled (e.g., polyethylene's minimum density is known to be approximately 0.85, the density of amorphous polyethylene; similar theoretical minimum densities are known or can be determined for different polymers, depending upon the identity of the polymer being modeled, as is known to those skilled in the art), nCH₂ is the amount (number) of methylene (CH₂) units in the component i (e.g., polymer backbone length in terms of number of CH₂ units), k is defined as provided in EQ. 8 below, and ρₘₐₓ (maximum density) is defined as provided in EQ. 9 below:

$$k = \frac{α}{{{log}_{10} ( nButyl + 2 )}^{β}} \qquad EQ . 8$$

where α and β are model parameters estimated by fitting this equation to density data obtained from samples of the polymer type being modeled and nButyl is the number of butyl branches in the component/(noting that incorporation of 1-hexene in a polyethylene backbone results in a 4-carbon SCB, that is a butyl branch). EQ. 8 can readily be adjusted for 1-butene comonomer by referencing ethyl branches; or for 1-octene comonomer by referencing hexyl branches; and so-on for other comonomer(s) incorporation with polyethylene monomer in a polymerization of ethylene and comonomer(s). And ρₘₐₓ is per EQ. 9:

$${ρ}_{max} = c 1 - c 2 {log}_{10} MW \qquad EQ . 9$$

where c1 and c2 are model parameters (numerical constants) estimated empirically from density data. For use of EQ. 9 and calculation of ρₘₐₓ, the method described in paragraphs [0123]-[0124] of US20110035193 (the '193 Publication) may be utilized, as exemplified for PE homopolymers in paras. [0125]-[0127] of the '193 Publication, all of the foregoing passages of which are incorporated herein by reference.

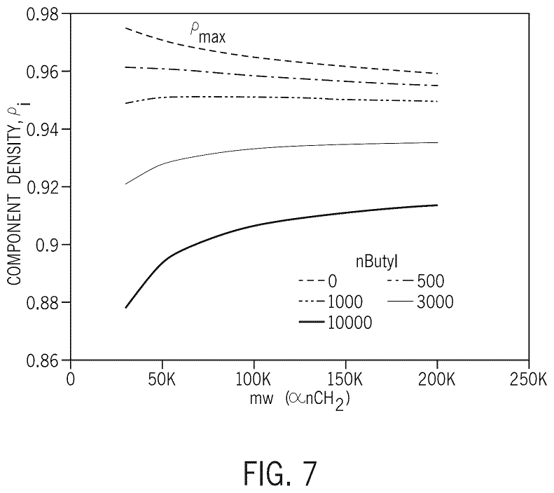

As indicated by FIG. 7, which is a graph of EQ. 7, EQ. 7 is consistent with the two PE density fundamentals trends described above. For example, as shown in FIG. 7, as the molecular weight increases, density deceases. Furthermore, as branching (as indicated by various values of nButyl in this particular example) increases, density decreases. In other words, the more comonomer incorporated into the component i, the lower the density of the component i.

Regarding melt index, the polymer trends may include a third trend and a fourth trend. The third trend may be that as molecular weight increases, the coil size of a polymer chain increases (in a melt), thereby resulting in a decreasing melt index. In other words, the higher the molecular weight, the lower the melt index. The fourth is that increased branching reduces the coil size of the polymer chain (in a melt) thereby resulting in increased melt index. That is, as branching increases, melt index increases. More specifically, higher amounts of long chain branching (e.g., relative to the amount of short chain branching) may lead to larger decreases in melt index.

Bearing this in mind, melt index of a component i may be modeled as provided in EQ. 10:

$$EQ . 10$$

$$ln ( {MI}_{i} ) = a - b * ( {MW}_{backbone} + {MW}_{branch} ) + c * {MW}_{backbone} * {MW}_{branch}$$

where the backbone and branch molecular weights can be readily calculated using the model generated at process block 62, and a, b, and c are model parameters determined from measured MI data on samples of polymer being modeled (noting that the more samples measured, the more accurate a, b, and c will be); preferably the samples will have varying comonomer content and molecular weight distributions.

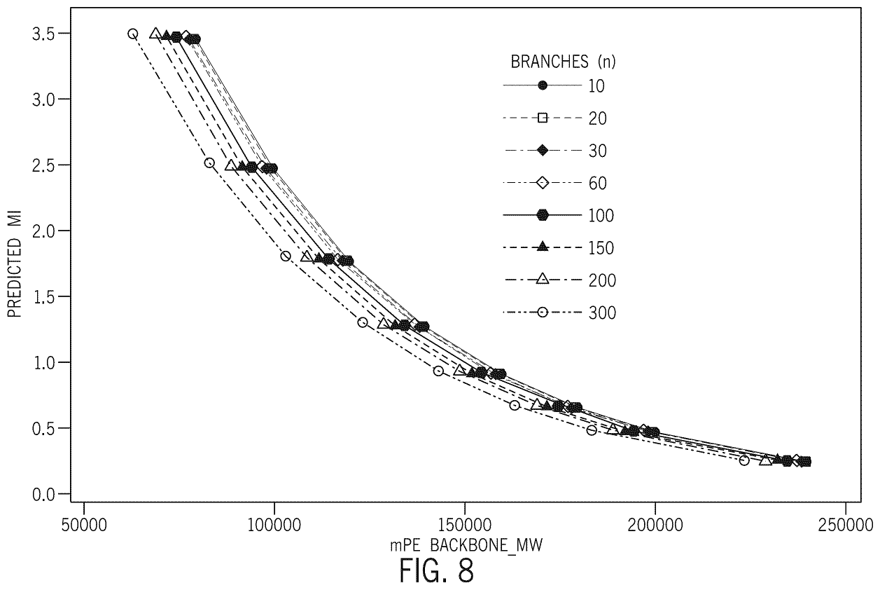

As indicated by FIG. 8, which is a graph of EQ. 10 (plotting predicted melt index vs. molecular weight of backbones of polyethylenes with varying amount of branching), EQ. 11 is consistent with the third trend and the fourth trend. Indeed, as shown in FIG. 8, as the molecular weight increases, the melt index decreases. Additionally, as the amount of branching increases (e.g., when comparing polymers having the same or relatively similar molecular weights), so does the melt index. As such, as demonstrated by EQ. 7-EQ. 10, FIG. 7, and FIG. 8, the density and melt index ratio of polymer components (e.g., polymers that are included in a polymer resin) may be modeled so that density and MI of polymers can be predicted from a limited data set of sample polymers. As discussed below, the properties of a polymer resin may accordingly be modeled.

Referring back to FIG. 4 and continuing with the discussion of the process 60, at process block 66, a model relating polymer component characteristics to polymer (resin) properties and/or polymer film properties (e.g., of a film made from polymer resin) may be generated. In particular, the polymer resin properties may include density, melt index, and melt index ratio. As discussed above, melt index and melt index ratio may also be referred to as melt flow rate (MFR) and melt flow rate ratio (MFRR), respectively. Polymer film properties are described below after discussing polymer resin properties. The model generated at process block 66 may be included in the model(s) generated at process block 62 and/or process block 64. Accordingly, by performing the operations associated with process block 62, 64, and/or 66, a single model may be generated.

Polymer resin properties may be determined based on one or more component properties (for each component of the polymer resin) as well as the relative abundance (e.g., weight percent) of each component. For example, the density of a polymer resin may be modeled according to EQ. 11:

$${ρ}_{polymer} = {( {\sum }_{i = 1}^{n} \frac{{w}_{i}}{{p}_{i}} )}^{- 1} \qquad EQ . 11$$

where ρ_{polymer} is the density of a polymer resin, w; is the weight percent of a component i in the polymer resin, and ρᵢ is the density of the component i. (e.g., as modeled as described above in connection with EQ. 7). As such, the density of a polymer resin may be determined by determining the inverse of the sum of a quotient for each polymer component of the polymer resin, with the quotient being the weight percent of the component in the polymer resin divided by the density of the component of the polymer resin.

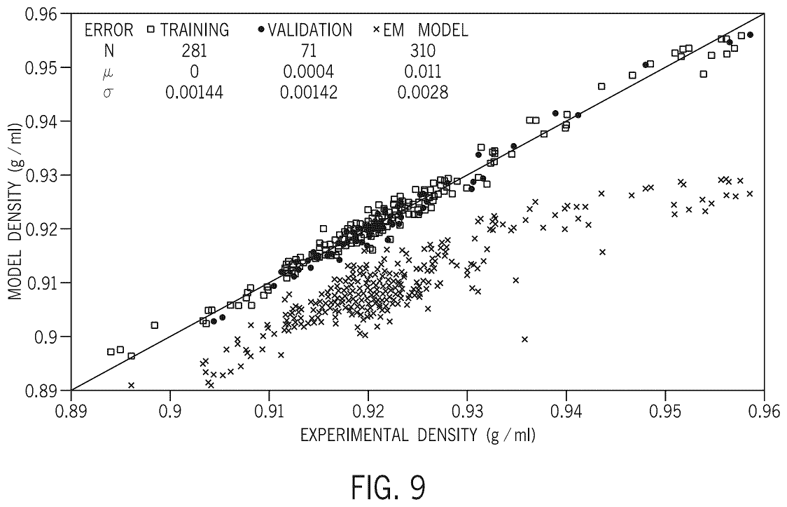

FIG. 9 is a graph plotting parity of predicted density vs. actual density between the model in accordance with EQ. 11 (as indicated by “training” and “validation”) and a pure machine learning correlation (“EM Model”). Utilizing EQ. 11 to model polymer resin density may enable polymer resin densities to be predicted for relatively large ranges of densities. Moreover, as evidenced by FIG. 9, using the model described herein may more accurately predict density relative to other techniques, such as models that only utilize machine learning.

Another polymer resin property that may be modeled is melt index. For example, as provided in EQ. 12 below, the melt index of a polymer resin may be modeled as a function of the melt index of the components (e.g., as modeled as described above in connection with EQ. 10) and the corresponding weight percentages of the components in the polymer resin:

$$ln ( {MI}_{polymer} ) = {\sum }_{i = 1}^{n} {wt}_{i} * ln ( {MI}_{i} ) \qquad EQ . 12$$

where MI_{polymer} is the melt index of the polymer resin, wtᵢ is the weight percent of component i in the polymer resin, and MIᵢ is the melt index of the component i.

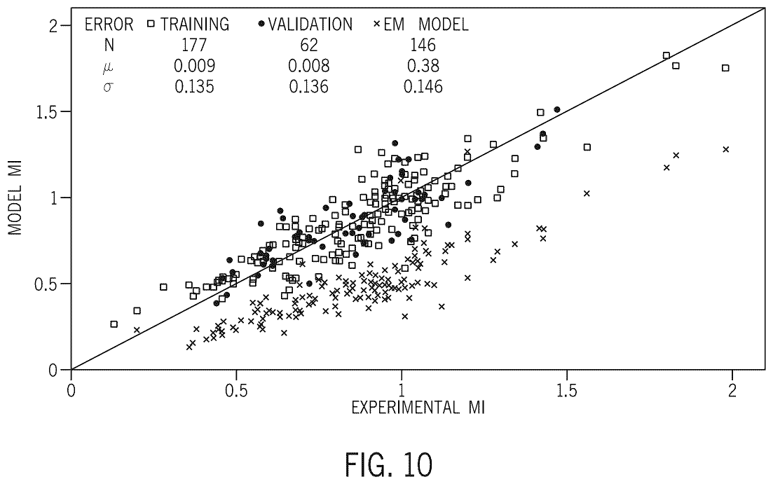

FIG. 10 is a graph plotting parity of predicted melt index vs. actual melt index between the model in accordance with EQ. 12 (as indicated by “training” and “validation”) and a pure machine learning correlation (“EM Model”). Utilizing EQ. 12 to model polymer resin density may enable polymer resin melt indices to be predicted for a relatively broad range of melt index values. Moreover, as evidenced by FIG. 10, using the model described herein may more accurately predict melt index relative to other techniques, such as models that only utilize machine learning.

Another polymer resin property which may be modeled at process block 66 is melt index ratio.

$$MIR = ( - 3.57 + 11.17 * ( \frac{{M}_{z}}{{M}_{w}} ) ) * {( \frac{{M}_{z}}{{M}_{n}} )}^{2} \qquad EQ . 13$$

where MIR is the melt index ratio, M_{Z} is the z-average molecular weight of the polymer resin, M_{W} is the weight average molecular weight of the polymer resin, and Mₙ is the number average molecular weight of the polymer resin.

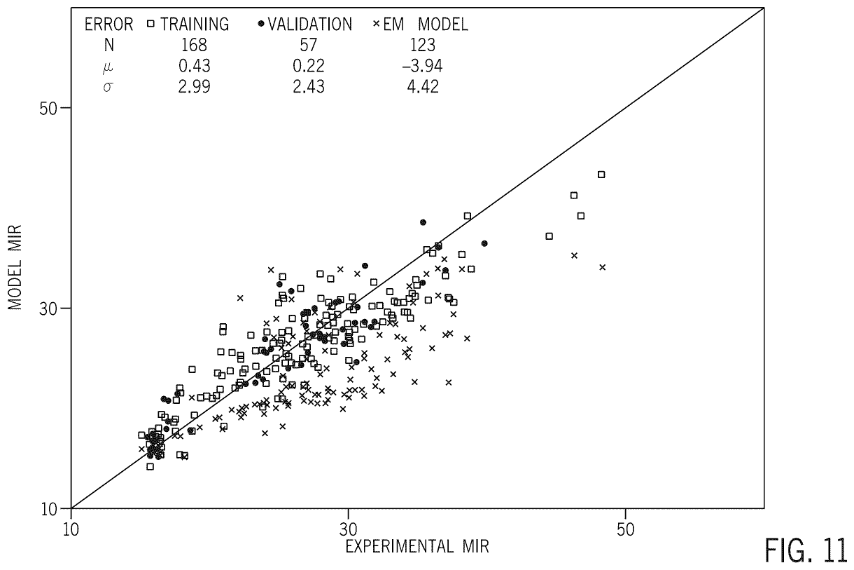

FIG. 11 is a graph plotting parity of predicted melt ratio index vs. actual melt index ratio between the model in accordance with EQ. 13 (as indicated by “training” and “validation”) and a pure machine learning correlation (“EM Model”). Utilizing EQ. 13 to model polymer resin density may enable polymer resin melt indices to be predicted for a relatively broad range of melt index values. Moreover, as evidenced by FIG. 11, using the model described herein may more accurately predict melt index relative to other techniques, such as models that only utilize machine learning.

The techniques herein may also be utilized to relate polymer component characteristics to polymer film properties, for example, by incorporating modeling techniques discussed above (e.g., in which polymer properties are related to polymer film properties). For example, for fixed fabrication settings (e.g., in which frost line height, temperature of polymer melt, and output rate are constant (or near constant)), polymer film properties may be predicted as discussed above with respect to EQ. 6 as well as FIG. 3A and FIG. 3B. Examples of the polymer film properties that may be predicted or modeled include secant modulus (e.g., one or both of machine direction secant modulus and transverse direction secant modulus), dart impact strength, haze total, tear (e.g., one or both of machine direction tear and transverse direction tear), tensile strength (e.g., one or both of machine direction tensile strength and transverse direction tensile strength), puncture break force, puncture break energy, break elongation (e.g., in one or both the machine and transverse directions), or any combination thereof. As such, the one or more models may relate polymer film properties to polymer component characteristics (e.g., properties), especially at fixed fabrication settings.

Referring back to FIG. 4 and the process 60, at process block 68, an input regarding target polymer properties and/or target polymer film properties may be received. For example, a computing device implementing the algorithms or models of the present disclosure may receive a user input indicative of target polymer properties of a polymer to be generated and/or target polymer film properties of a polymer film to be generated. The target polymer properties may include density, melt index, melt index ratio, or any combination thereof. The target polymer film properties may include secant modulus (e.g., one or both of machine direction secant modulus and transverse direction secant modulus), dart impact strength (e.g., a value in g/mil), haze total, tear (e.g., one or both of machine direction tear and transverse direction tear), tensile strength (e.g., one or both of machine direction tensile strength and transverse direction tensile strength), puncture break force, puncture break energy, break elongation (e.g., in one or both the machine and transverse directions), or any combination thereof.

At process block 70, polymer component parameters may be generated based on the target polymer properties and/or the target polymer film properties indicated by the input received at process block 68. For example, the model(s) associated with process block 62, 64, and 66 may include one or more algorithms that utilize a user input (e.g., target polymer properties and/or target polymer film properties) to determine or generate polymer component parameters to result in a polymer (e.g., resin or film) with the target properties. Accordingly, the algorithm(s) may be derived from the model(s) described above, and the algorithm(s) may include EQS. 7-13.

As an example, in which the input includes one or more target polymer properties (e.g., density, melt index, melt index ratio, or any combination thereof), using the model-derived algorithm(s), the polymer component parameters may be generated. In particular, the polymer component parameters may include the number of methylene (CH₂) units in each polymer component, the number of hexene units (or other comonomer units) in each polymer component, and the weight percent of each polymer component (e.g., among the polymers of a polymer resin). As such, the polymer component parameters may include one or more polymer components that make up a polymer to be generated (e.g., a polymer resin that includes one or more polymer ensembles that include one or more polymer components) as well as characteristics of the polymer components. As described above, properties of polymers (e.g., resins) as well as polymer films may be modeled and related to the polymer components. As such, particular polymer component parameters may be determined such that when the corresponding polymer components are generated, the resulting polymer (or polymer) has properties equal to or similar to (e.g., within 5%, within 2%, within 1%) the target properties.

At process block 72, a polymer is generated using the generated polymer component parameters. For example, the polymer may be polyethylene that may be generated in a GPPE reactor. In this manner, a polymer having polymer properties that are equal or approximately equal (e.g., within 5%, within 2%, within 1%) to the target polymer properties (e.g., as specified in a user input received at process block 68) may be generated. The polymer generated in process block 72 may be a polymer resin that includes several polymers (e.g., polymer components). The amounts of the polymer components may be those determined at process block 70 or approximately equal (e.g., within 5%, within 2%, within 1%) to those determined at process block 70.

Moreover, the polymer may be extruded (e.g., using blown film extrusion) to produce a polymer film having polymer film properties that are equal to or substantially equal to the target polymer film properties. To do so, the polymer may be heated to generate molten polymer, and the molten polymer may be extruded using the blown film extrusion properties discussed above.

Polymers, including polyethylene, may be made by any suitable polymerization method including solution polymerization, slurry polymerization, supercritical, and gas phase polymerization using supported or unsupported catalyst systems, such as a system incorporating a metallocene catalyst.

In accordance with the present application, a method for producing a polymer includes generating polymer component parameters using a model comprising an algorithm with an input of target polymer properties of the polymer. The target polymer properties include a density of the polymer, a melt index of the polymer, and a melt index ratio of the polymer. The polymer includes a plurality of polymer molecules, and the polymer component parameters include respective amounts of respective polymer molecules of the plurality of polymer molecules. The method also includes producing the polymer using the polymer component parameters.

In the method, the polymer may include a copolymer of ethylene and hexene. In the method the polymer component parameters may also include respective amounts of methylene units of the respective polymer molecules. Moreover, the polymer component parameters may include respective amounts of long chain branches of the respective polymer molecules as well as respective amounts of hexene-derived units of the respective polymer molecules.

In the method, generating the polymer component parameters may include determining a polymer density of the polymer based on a plurality of densities of the plurality of polymer molecules. The plurality of densities are determined using EQ. 7 (as described above), with references to EQ. 8 (determination of k) and EQ. 9 (determination of ρₘₐₓ) as also described above.

In the method, generating the polymer component parameters may include determining the melt index of the polymer based on a plurality of melt indexes of the plurality of polymer molecules. The plurality of melt indexes are determined using EQ. 10 as described above.

In accordance with the present application, a method for producing a polymer includes generating polymer component parameters using a model comprising an algorithm with an input of target polymer properties of a polymer film. The polymer film includes the polymer, and the polymer includes a plurality of polymer molecules. The polymer component parameters include respective amounts of respective polymer molecules of the plurality of polymer molecules. The method also includes producing the polymer using the polymer component parameters.

In the method, the polymer may include a copolymer of ethylene and hexene. Additionally, the target polymer film properties may include a machine direction secant modulus, a transverse direction secant modulus, a dart impact strength, a haze total, a machine direction tear, a transverse direction tear, a machine direction tensile strength, a transverse direction tensile strength, a puncture break force, a puncture break energy, or any combination thereof. In the method, the target polymer film properties may include at least three of the machine direction secant modulus, the transverse direction secant modulus, the dart impact strength, the haze total, the machine direction tear, the transverse direction tear, the machine direction tensile strength, the transverse direction tensile strength, the puncture break force, or the puncture break energy. In the method, the target polymer film properties may include at least three of the machine direction secant modulus, the transverse direction secant modulus, the dart impact strength, the haze total, the machine direction tear, the transverse direction tear, the machine direction tensile strength, the transverse direction tensile strength, the puncture break force, and the puncture break energy.

In the method, the polymer component parameters may include respective amounts of methylene units of the respective polymer molecules, respective amounts of long chain branches of the respective polymer molecules, respective amounts of hexene-derived units of the respective polymer molecules, or any combination thereof.

Additionally, the method may include generating one or more polymer properties based on the target polymer film properties and producing the polymer component parameters based on the one or more polymer properties. Moreover, the one or more polymer properties may include a density, a melt index, a melt index ratio, or any combination thereof. In the method, producing the polymer may include producing the polymer using at least one metallocene catalyst.

In accordance with the present application, a computing device (e.g., for controlling a polymer production system) includes a processor, a memory coupled to the processor, and instructions provided to the memory. The instructions are executable by the processor to perform any method or process described herein.

### Relating Cross-Fractionation Characterizations to Polymer Properties

To accelerate the design process of new polymers, such as (but not limited to) linear low density polyethylene, models can be utilized which connect product composition to end-use properties. Such models may use the input of a composition distribution for the polymer (e.g., a LLDPE product) and generate an output that may include end-use properties of the polymer and/or a polymer film produced from the polymer. The composition distribution may be a two-dimensional distribution, such as with a Cross-Fractionation Characterization (CFC). However, such a model input may be difficult to hypothetically devise in advance, for example, because the scale-up to pilot reactors for larger-scale property testing may be the only portion of the design process circumvented. In other words, the polymer would still first need to be produced. Subsequently, a CFC of the polymer would be generated, and the CFC could be used as an input into a model. To fully circumvent a design iteration, making a completely virtual product design process possible, the techniques described herein may be utilized. In particular, a hypothetical CFC may be generated using parameters or characteristics set by a user input. A functional form is assumed for each peak in a CFC, and several parameters are used to describe each peak using the function. Multiple peaks can be added together to compose the entire hypothetical CFC. Using composition-property models (such as those described above with respect to the process 60) in tandem with these hypothetical CFC constructions, a product designer can iterate on LLDPE compositions instantaneously, and experimentally validate at a later stage, once a desired composition distribution (e.g., a desired hypothetical CFC construction) is output based upon input of desired polymer and/or film properties. This design approach could reduce the number of physical iterations necessary to develop a polymer product having desired properties, and/or a polymer product that would produce a film of desired film properties. Moreover, these techniques may be combined with the process 10 in order to determine reactor parameters to use to produce polymers.

Before discussing the techniques of the present disclosure in more detail, it should be noted that while the techniques of the present disclosure are discussed below with respect to LLDPE products, the present techniques are not limited to such products. In other words, LLDPE products (and the production thereof) are provided as an example context for which the techniques of the present disclosure may be utilized. The design of a new polymer (e.g., a LLDPE product) typically involves selecting a catalyst system and/or a synthesis method in order to produce a reliable, unique composition of polymer (e.g., polyethylene) with desirable end-use properties. For some polymers, such ethylene-olefin LLDPE copolymers such as ethylene-hexene, this unique composition can be defined for instance by CFC. CFC measures the weight distribution for polydisperse LLDPE across a range of molecular weights and solvent elution temperatures, which corresponds to a comonomer incorporation percentage as a function of chain length (molecular weight) of the polymer chain population within the polymer composition. High elution temperature maps to low comonomer incorporation in polymer chain(s) of corresponding molecular weight, and low elution temperature maps to high comonomer incorporation in polymer chain(s) of corresponding molecular weight.

A CFC distribution is a unique and complete description of a LLDPE product. Because of this, it may be beneficial to consider a new product's CFC signature during its design. For instance, relationships have been established between CFC signatures and film properties; conventional composition distributions (polyethylene whose high molecular weight components have a high elution temperature and whose low molecular weight molecules have a low elution temperature) yield relatively high film tear resistance, while broad orthogonal composition distributions (polyethylene whose high molecular weight components have a low elution temperature, that is a high comonomer incorporation and whose low molecular weight molecules have a high elution temperature, that is a low comonomer incorporation) yield relatively high film dart drop resistance. These manners of property distinctions hold true even at the same density and melt index, which are otherwise generally the primary specifiers for polyethylene products.

As an example, and to add more context, a typical metallocene LLDPE (mLLDPE) design process may progress by selecting a synthesis method (e.g. a gas phase reaction or a solution phase reaction), then identifying and/or inventing an appropriate catalyst to use for the synthesis that may give the desired properties (e.g. a Ziegler-Natta catalyst or one of many varieties of metallocene catalyst). Significant development time will be invested in establishing adequate reactor conditions and generating small-scale quantities of product to validate reaction feasibility. Afterwards, sufficient quantities of the experimental product can be created to be converted into articles like film, for testing for end-use properties. The results can then be compared against the designer's intuition and expectation, and adjustments may be made for the next iteration of the cycle.

Such a process can be circumvented by building models connecting composition to properties. As described herein, the present disclosure relates to a model that receives an experimental CFC distribution as the sole input and predicts a variety of polymer properties (including density, melt index), and/or film mechanical and optical properties (for a hypothetical film made from the polymer). The model is valid for polymers including, but not limited to, polyethylene, such as polyethylene copolymers like LLDPE copolymers (such as ethylene-hexene, ethylene-butene, and/or ethylene-octene LLDPEs). Ethylene-hexene LLDPEs are used herein to exemplify the process.

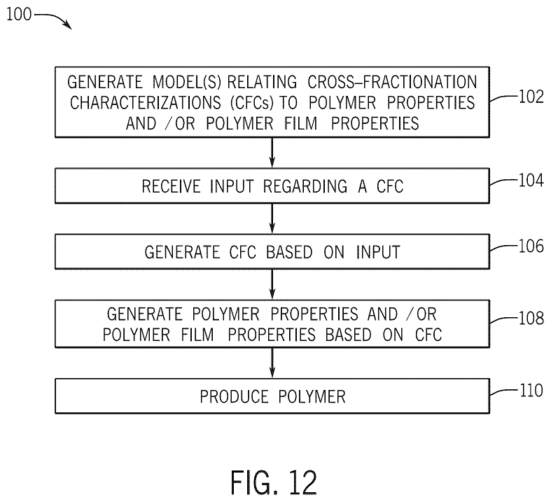

Bearing this in mind, FIG. 12 is a flow diagram of a process 100 for producing a polymer, such as polyethylene. The process 100 may be performed on one or more computing devices (or processor-based devices) that include a processor, a memory coupled to the processor, and instructions provided to the memory. The instructions are executable by the processor to perform the methods and/or processes described herein. The instructions can be a portion of code on a non-transitory computer readable medium. Any suitable processor-based device may be utilized for implementing all or a portion of embodiments of the present techniques, including without limitation personal computers, networks personal computers, laptop computers, computer workstations, mobile devices, multi-processor servers or workstations with (or without) shared memory, high performance computers, and the like. Moreover, embodiments may be implemented on application specific integrated circuits (ASICs) or very large scale integrated (VLSI) circuits. The process 100 generally includes generating one or more models relating a cross-fractionation characterization (CFC) to polymer properties and/or polymer film properties (process block 102), receiving an input regarding a CFC (process block 104), generating the CFC based on the input (process block 106), generating polymer properties and/or polymer film properties based on the CFC (process block 108), and producing the polymer (process block 110). The polymer may have the polymer properties or be used to make a film having the polymer film properties.

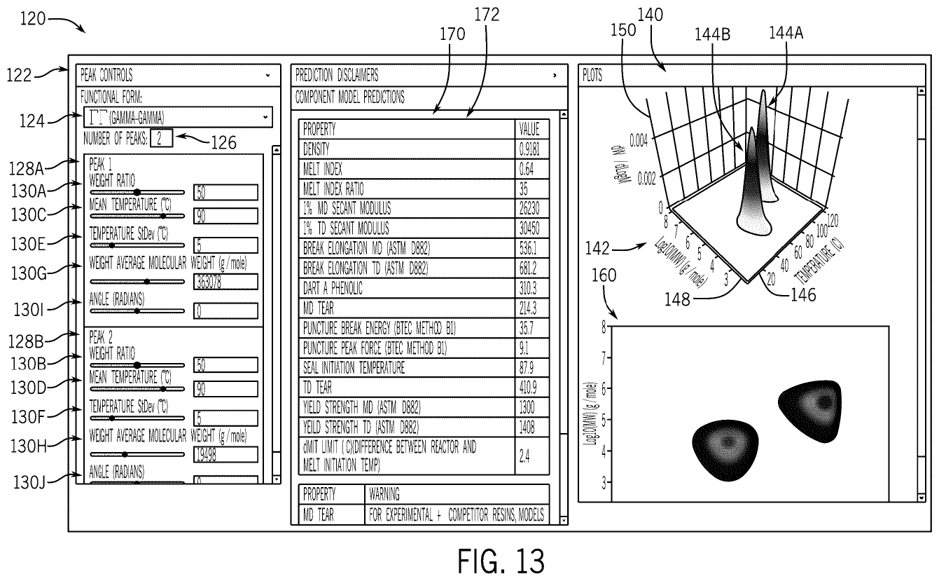

At process block 102, one or more models relating cross-fractionation characterizations (CFCs) to polymer properties and/or polymer film properties may be generated. The one or more models may utilize machine learning and may be specific to a particular polymer (e.g., a LLDPE or mLLDPE). Modeling and the generation of CFCs will be discussed in more detail below with respect to process block 106 of the process 100 as well as FIG. 13.

At process block 104, an input regarding a CFC may be received. For example, referring to FIG. 13, which is a graphical user interface (GUI) 120 (e.g., of modeling software) that may be displayed on a computing system used to perform the process 100 or a portion thereof, a user may interact with an editing portion 122 of the user interface 120 to select a functional form using GUI element 124 (e.g., a dropdown menu), a number of peaks in a CFC (when generated) using GUI element 126, and characteristics of the peak(s) of the CFC using the peak sections 128 (which collectively refers to peak section 128A and peak section 128B) of the GUI 120. While the GUI 120 includes two peak sections 128 in FIG. 13, there may be as many peak sections 128 as there are peaks in the GUI element 126. Each of the peak sections 128 include several GUI elements 130 (which may include a slider and/or an entry field (that may include a numerical value) that a user may interact with to select or alter characteristics of the peak(s) of the CFC. More specifically, as illustrated, the GUI elements 130 include GUI element 130A and GUI element 130B for editing weight ratio, GUI element 130C and GUI element 130D for editing mean temperature, GUI element 130E and GUI element 130F for editing standard deviation of the mean temperature, GUI element 130G and GUI element 130H for editing weight average molecular weight, and GUI element 130I and GUI element 130J for angle. In particular, a user may add a (single) rotation angle parameter to any of the single mode functional expressions, introducing one more degree of freedom to each mode. The rotation can be performed about the mean temperature and molecular weight of a mode using a two-dimensional rotation transformation.

The functional form (selectable using the GUIitem 124) may be one of three functional forms that can be utilized to generate a CFC (e.g., a CFC generated at process block 106, as discussed below). In other embodiments, there may be fewer than three (e.g., one or two) functional forms that may be selected using the GUI item 124, while in other embodiments, more than three functional forms (e.g., four, five, six, or more) may be selectable via the GUI item 124. Several functional forms and related equations are discussed below. A first functional form may be a gamma-Flory/Schulz form that utilizes EQ. 14:

$$\frac{dw ( M , F )}{d {log}_{10} M dF} = ln 10 \frac{{M}^{2}}{{θ}_{m}^{2}} {e}^{- M / {θ}_{m}} \times \frac{{F}^{( {k}_{f - 1} )}}{Γ ( {k}_{f} ) {θ}_{f}^{{k}_{f}}} {e}^{- F / {θ}_{f}} \qquad EQ . 14$$

where w is weight, M is molecular weight, F is comonomer mole percent, θₘ and θ_{f} are respectively characteristic values for M and F, k_{f} is a distribution breadth parameter, and Γ is a gamma function. In the first functional form, the number-averaged molecular weight (Mn) is equal to the characteristic θₘ. Additionally, the weight-averaged molecular weight (Mw) is equal to two times the characteristic θₘ. Accordingly, the ratio of the weight-averaged molecular weight to the number-averaged molecular weight, commonly referred to as the polydispersity index or PDI, may be equal to two. Furthermore, the moments of the comonomer distribution for the first functional form are described by EQ. 15 and EQ. 16 as set forth below:

$${μ}_{f} = {k}_{f} {θ}_{f} \qquad EQ . 15$$

$${σ}_{f} = \sqrt{{k}_{f}} {θ}_{f} = \frac{{μ}_{f}}{\sqrt{{k}_{f}}} \qquad EQ . 16$$

where μ_{f} is the mean comonomer mole percent, and of is the standard deviation of the comonomer mole percent.

A second functional form, which may be a gamma-gamma form (e.g., as shown in the GUI item 124 of FIG. 13), may utilize EQ. 17:

$$\frac{dw ( M , F )}{d {log}_{10} M dF} = ln 10 \frac{{M}^{{k}_{m}}}{Γ ( {k}_{m} ) {θ}_{m}^{{k}_{m}}} {e}^{- M / {θ}_{m}} \times \frac{{F}^{( {k}_{f - 1} )}}{Γ ( {k}_{f} ) {θ}_{f}^{{k}_{f}}} {e}^{- F / {θ}_{f}} \qquad EQ . 17$$

where kₘ is a distribution breadth parameter. The molecular weight averages for the second functional form are provided below in EQS. 18-20:

$${M}_{n} = ( {k}_{m} - 1 ) {θ}_{m} \qquad EQ . 18$$

$${M}_{w} = {k}_{m} {θ}_{m} \qquad EQ . 19$$

$$PDI = \frac{{k}_{m}}{{k}_{m} - 1} \qquad EQ . 20$$

where Mₙ is the number-averaged molecular weight, and M_{w} is the weight-averaged molecular weight. As discussed above, PDI is the ratio of the weight-averaged molecular weight to the number-averaged molecular weight. In the second functional form, the PDI may be equal to a value between one and twenty, inclusive. For example, the PDI may be set equal to one, two, three, or any value between one and three, inclusive. Additionally, the second functional form may utilize EQ. 15 and EQ. 16 as set forth above.

A third functional form, which may be a Pearson IV-Pearson IV functional form, may utilize EQS. 21-23:

$$\frac{dw ( M , T )}{d {log}_{10} M dT} = \frac{{e}^{- {S}_{m} {tan}^{- 1} \hat{M}}}{{( 1 + {\hat{M}}^{2} )}^{{K}_{m}}} \times \frac{{e}^{- {S}_{t} {tan}^{- 1} \hat{T}}}{{( 1 + {\hat{T}}^{2} )}^{{K}_{t}}} \qquad EQ . 21$$

$$\hat{M} = \frac{{log}_{10} M - {L}_{m}}{{W}_{m}} \qquad EQ . 22$$

$$T = \frac{T - {L}_{t}}{{W}_{t}} \qquad EQ . 23$$

where T is temperature, Lₘ and Lₜ are respective location parameters for the molecular weight and temperature distributions, Wₘ and Wₜ are respective width parameters for the molecular weight and temperature distributions, Sₘ and Sₜ are respective skewness parameters for the molecular weight and temperature distributions, and Kₘ and Kₜ are respective kurtosis parameters for the molecular weight and temperature distributions.

In the functional forms, the comonomer mole percent and temperature may be directly related in a CFC experiment through a calibration function, which may be a linear fit. If the calibration produces instances in which monomer mole percent is less than zero at a valid temperature, the value of the comonomer mole fraction may be set to zero (instead of using a negative value).

Returning to FIG. 12 and continuing with the discussion of the process 100, at process block 106, one or more CFC may be generated based on the input (as received at process block 104). For example, a computing device or system may utilize one or more of EQ. 14-23 to generate one or more CFCs based on values and the functional form as defined by the input received at process block 104. For example, in the context of FIG. 13, the GUI 120 may include a CFC section 140 in which a CFC 142 may be generated and presented in one or more perspectives. For example, the CFC 142 may be presented three-dimensionally and includes peaks 144 (collectively referring to peak 144A and peak 144B, which respectively correspond to the first and second peak in the editing portion 122 of the GUI 120), with axis 146 (temperature), axis 148 (molecular weight (logarithmic)), and axis 150 (dW/d Log M, with W being weight and M being molecular weight). The CFC 142 may also be presented two-dimensionally, such as in CFC 160, which also includes the peaks as well as axes for temperature and molecular weight (logarithmic).

Multiple modes can be included in a single CFC signature by giving each mode a relative amplitude, a multiplicative factor to the function, then adding the values for all modes together at each molecular weight and temperature of interest. By computing this aggregate function for an entire range of temperature and molecular weight (or equivalently the comonomer mole percent and molecular weight), the entire CFC (e.g., CFC 142 and/or CFC 144) can be constructed. This in turn can be fed into component-based property models for prediction.

There is additional utility in carefully choosing a functional form that has some theoretical basis in reaction kinetics. For example, the first functional form can be linked to kinetic rate constants for general coordination insertion polymerization. By designing a hypothetical CFC using this functional form, the potential rate constants and reaction conditions can be derived from the CFC, which can guide in catalyst and process design efforts.

Returning to FIG. 12 and the discussion of the process 100, at process block 108, polymer properties and/or polymer film properties based on the CFC (as generated at process block 106). For example, the CFC may be used as an input into a component-based model, such as the model used in the process 60. More specifically, the CFC may be utilized to define polymer component parameters, and the model may be used to generate polymer properties and/or polymer film properties using the polymer component parameters (as determined using, or defined by, the CFC).

As illustrated in FIG. 13, the GUI 120 may include a property section 170, which may name polymer properties and/or polymer film properties as well as values of the properties. For example, the property section 170 may include a table 172 that lists the polymer properties, polymer film properties, and values of the polymer properties and the polymer film properties. As illustrated, the polymer properties may include density, melt index, and melt index ratio. The polymer film properties may include one or more of: secant modulus (e.g., one or both of machine direction secant modulus and transverse direction secant modulus), dart impact strength, haze total, tear (e.g., one or both of machine direction tear and transverse direction tear), tensile strength (e.g., one or both of machine direction tensile strength and transverse direction tensile strength), puncture break force, puncture break energy, break elongation (e.g., in one or both the machine and transverse directions), yield strength (e.g., in one or both the machine and transverse directions), and a melt initiation temperature (or a value derived from a melt initiation temperature).

By utilizing functional forms for CFCs with relatively limited degrees of freedom per mode (e.g., 3-5 degrees of freedom), dynamic adjustment of the composition and even optimization is possible. For example, as a user views the GUI 120, the user may modify or provide additional inputs in the editing portion 122 of the GUI 120, and the CFC 242 (and CFC 244) as well as the values in the table 172 of the property section 170 of the GUI 120 will update (e.g., providing values for a prediction updated based on the additional input(s)). It should be noted that, in other embodiments, other algorithms that utilize more degrees of freedom may be applied to maximize an objective function corresponding to a desired set of end-use properties.

Returning to FIG. 12 and the discussion of the process 100, at process block 110, the polymer may be produced. In other words, a polymer which may have polymer components corresponding to the CFC 242 (and CFC 244) and the polymer properties (e.g., within one percent, three percent, or five percent) determined (at process block 108) and provided in the property section 170 of the GUI 120. Additionally, a polymer film may be made from the polymer, and the polymer film may have (e.g., within one percent, three percent, or five percent) the polymer film properties as previously determined (and provided in the GUI 120).

As an example, to produce the polymer, the polymer properties and/or polymer film properties may be utilized as the input when performing the process 10 (e.g., at process block 14). Reactor parameters may be determined based on the desired polymer properties and/or polymer film properties, as discussed above. Accordingly, the process 100 may include one or more portions of the process 10 as well as the process 60. Furthermore, kinetic parameters may also be determined. That is, a catalyst possessing desired kinetic parameters can be utilized in combination with the reactor conditions, which are part of reactor parameters, to produce the polymer. For example, potential kinetic parameters and reactor parameters can be determined using a polymer structure distribution model (e.g., Stockmayer bivariate distribution model or other type distribution models) and a parameter estimation model. Once the desired polymer structure (e.g., in a CFC) is determined (i.e. the set of distribution constants for example in EQS 14-23), it can be used to extract kinetic and reactor information such as ratio of rate constants, ratio of rates, average comonomer percent, etc. The parameter estimation model is then used to identify the potential kinetic parameters and reactor parameters that will satisfy the specified polymer distribution.

The polymer produced per the above description may be extruded (e.g., using blown film extrusion) to produce a polymer film having polymer film properties that are equal to or substantially equal to the target polymer film properties. To do so, the polymer may be heated to generate molten polymer, and the molten polymer may be extruded using the blown film extrusion properties discussed above.

Polymers, including polyethylene, may be made by any suitable polymerization method including solution polymerization, slurry polymerization, supercritical, and gas phase polymerization using supported or unsupported catalyst systems, such as a system incorporating a metallocene catalyst.

In accordance with present embodiments, a method for producing a polymer includes generating polymer properties of the polymer using a model that includes an algorithm with an input of a cross-fractionation characterization (CFC) of the polymer. The CFC is generated based on a user input regarding one or more portions of the CFC. Additionally, the method includes producing the polymer having the polymer properties. The polymer may still be considered to have the polymer properties when the polymer has polymer properties within a threshold range (e.g., within one percent, three percent, five percent) of the polymer properties.

In the method, the one or more portions of the CFC may correspond to one or more peaks of the CFC. Additionally, the one or more peaks may include a first peak and a second peak that respectively correspond to a first component and a second component of the polymer. The user input may include a first weight ratio of the first component, a second weight ratio of the second component, a first molecular weight of the first component, and a second molecular weight of the second component. Moreover, the user input may include a first elution temperature of the first component and a second elution temperature of the second component.

In the method, wherein the polymer properties comprise a density, a melt index, a melt index ratio, or any combination thereof. Additionally, the method may include generating reactor parameters using a second model that includes a Gaussian process model-derived algorithm with an input of the polymer properties of the polymer. The method may also include producing the polymer comprises producing the polymer using the reactor parameters. The reactor parameters comprise at least two of: a reactor bed temperature, a hexene to ethylene flow ratio, a hydrogen to ethylene gas ratio, a reactor residence time, a partial pressure of ethylene, or an isopentane composition. Furthermore, the polymer may include a plurality of polymer molecules, the method may also include determining polymer component parameters of the polymer, the polymer component parameters include respective amounts of respective polymer molecules of the plurality of polymer molecules, and generating the polymer properties includes generating the polymer properties based on the polymer component parameters.

In the method, the polymer may be or include a linear low-density polyethylene (LLDPE). Additionally, the method may include generating, based on the user input, the CFC in a graphical user interface.

In accordance with present embodiments, a method for producing a polymer includes generating polymer film properties of a polymer film using a model having an algorithm with an input of a cross-fractionation characterization (CFC) of the polymer. The polymer film includes the polymer, and the CFC is generated based on a user input regarding one or more portions of the CFC. Additionally, the method includes producing the polymer. The process may additionally include producing a polymer film from the polymer. When produced, the polymer film has the polymer film properties (or values for the polymer film properties within a threshold range (e.g., one percent, three percent, five percent) of the polymer film properties).

In the method, the polymer film properties may include any one or more of: a machine direction secant modulus, a transverse direction secant modulus, a dart impact strength, a haze total, a machine direction tear, a transverse direction tear, a machine direction tensile strength, a transverse direction tensile strength, a puncture break force, a puncture break energy, or any combination thereof. The polymer film properties may include at least three of the machine direction secant modulus, the transverse direction secant modulus, the dart impact strength, the haze total, the machine direction tear, the transverse direction tear, the machine direction tensile strength, the transverse direction tensile strength, the puncture break force, or the puncture break energy.

In the method, the one or more portions of the CFC may correspond to one or more peaks of the CFC, and the user input may define how many peaks are included in the one or more peaks.

The method may also include generating, using the model and based on the CFC, polymer properties of the polymer. The polymer properties comprise a density, a melt index, and a melt index ratio of the polymer.

In the method, the polymer may include a plurality of polymer molecules. Additionally, the method may include determining polymer component parameters of the polymer, and the polymer component parameters may include respective amounts of respective polymer molecules of the plurality of polymer molecules. Also, the method may include generating the polymer film properties comprises generating the polymer film properties based on the polymer component parameters. Moreover, the method may include generating reactor parameters based on the polymer film properties of the polymer and producing the polymer using the reactor parameters. The reactor parameters may include a catalyst composition having at least one metallocene catalyst as well as at least two of: a reactor bed temperature, a hexene to ethylene flow ratio, a hydrogen to ethylene gas ratio, a reactor residence time, a partial pressure of ethylene, or an isopentane composition.

In the method, wherein the polymer may be or include a metallocene linear low-density polyethylene (mLLDPE).

In accordance with the present application, a computing device (e.g., for controlling a polymer production system) includes a processor, a memory coupled to the processor, and instructions provided to the memory. The instructions are executable by the processor to perform any method or process described herein.

While only certain features of the disclosure have been illustrated and described herein, many modifications and changes will occur to those skilled in the art. It is, therefore, to be understood that the appended claims are intended to cover all such modifications and changes as fall within the true spirit of the disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses embodiments/examples to disclose the invention, including the best mode, and also to enable any person skilled in the art to practice the invention, including making and using any devices or systems and performing any incorporated methods. The patentable scope of the invention is defined by the claims, and may include other embodiments/examples that occur to those skilled in the art. Such other embodiments/examples are intended to be within the scope of the claims if they have structural elements that do not differ from the literal language of the claims, or if they include equivalent structural elements with insubstantial differences from the literal language of the claims. Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

Embodiment 1. A method for producing a polymer, the method comprising generating reactor parameters using a model comprising a Gaussian process model-derived algorithm with an input of target polymer properties of the polymer, wherein the reactor parameters comprise a catalyst composition having at least one metallocene catalyst; and producing the polymer using the reactor parameters.

Embodiment 2. The method of Embodiment 1, wherein the polymer comprises a polyethylene.

Embodiment 3. The method of Embodiment 2, wherein producing the polymer comprises producing the polyethylene using a gas phase polyethylene reactor operating using the reactor parameters.

Embodiment 4. The method of Embodiments 1-3, wherein the catalyst composition comprises a mixed catalyst composition comprising two or more metallocene catalysts.

Embodiment 5. The method of Embodiments 1-4, wherein the reactor parameters comprise a reactor bed temperature, a hexene to ethylene feed flow ratio, a hydrogen to ethylene gas ratio, a reactor residence time, a partial pressure of ethylene, an isopentane composition, or any combination thereof (or, more broadly, wherein the reactor parameters comprise any one or more of a reactor temperature, a comonomer to monomer feed flow ratio, a hydrogen to monomer gas ratio, a reactor residence time, a partial pressure of monomer, and an induced condensing agent (ICA) composition).

Embodiment 6. The method of Embodiment 5, wherein the reactor parameters comprise the reactor bed temperature, the hexene to ethylene feed flow ratio, the hydrogen to ethylene gas ratio, the reactor residence time, the partial pressure of ethylene, and the isopentane composition (or, more broadly, wherein the reactor parameters comprise the reactor temperature, the comonomer to monomer feed flow ratio, the hydrogen to monomer gas ratio, the reactor residence time, the partial pressure of monomer, and the induced condensing agent (ICA) composition.

Embodiment 7. The method of Embodiments 1-6, wherein the target polymer properties comprise a bulk density, a melt flow rate, a flow rate ratio, or any combination thereof.

Embodiment 8. The Embodiment of Embodiment 7, wherein the target polymer properties comprise the bulk density, the melt flow rate, and the flow rate ratio.

Embodiment 9. The method of Embodiments 1-3, wherein: the catalyst composition comprises a mixed catalyst composition comprising two or more metallocene catalysts; and the reactor parameters comprise a reactor bed temperature, a hexene to ethylene feed flow ratio, a hydrogen to ethylene gas ratio, a reactor residence time, a partial pressure of ethylene, an isopentane composition, or any combination thereof (or, more broadly, wherein the reactor parameters comprise a reactor temperature, a comonomer to monomer feed flow ratio, a hydrogen to monomer gas ratio, a reactor residence time, a partial pressure of monomer, an induced condensing agent (ICA) composition, or any combination thereof).

Embodiment 10. The method of Embodiment 1-9, wherein the Gaussian process model-derived algorithm comprises a kernel function of K(xᵢ, xⱼ)=σ² exp(−(xᵢ−xⱼ)ⁿ/2L²)+δᵢⱼσ²ₙₒᵢₛₑ, where xᵢ is a vector of prediction variables at experiment i, xⱼ is a vector of the prediction variables at experiment j, σ and Z are model hyper-parameters, δᵢⱼ is equal to zero when i and j and equal to one when i and j are equivalent, σ²ₙₒᵢₛₑ is a positive value, and n is a value ranging from one to five, inclusive.

Embodiment 11. A computing device for controlling a polymer production system, the computing device comprising: a processor; a memory coupled to the processor; and instructions provided to the memory, wherein the instructions are executable by the processor to perform the method of Embodiments 1-10.

Embodiment 12. A method for producing a polymer, the method comprising: generating reactor parameters using a model comprising a Gaussian process model-derived algorithm with an input of target polymer film properties of a polymer film comprising the polymer, wherein the reactor parameters comprise a catalyst composition having at least one metallocene catalyst; and producing the polymer using the reactor parameters.

Embodiment 13. The method of Embodiment 12, wherein the polymer comprises a polyethylene.

Embodiment 14. The method of Embodiment 12 or 13, wherein producing the polymer comprises producing the polyethylene using a gas phase polyethylene reactor operating using the reactor parameters.

Embodiment 15. The method of Embodiments 12-14, wherein the catalyst composition comprises a mixed catalyst composition comprising two or more metallocene catalysts.

Embodiment 16. The method of Embodiments 12-15, wherein the reactor parameters comprise a reactor bed temperature, a hexene to ethylene feed flow ratio, a hydrogen to ethylene gas ratio, a reactor residence time, a partial pressure of ethylene, and an isopentane composition (or, more broadly, wherein the reactor parameters comprise a reactor temperature, a comonomer to monomer feed flow ratio, a hydrogen to monomer gas ratio, a reactor residence time, a partial pressure of monomer, and an induced condensing agent (ICA) composition).

Embodiment 17. The method of Embodiments 12-16, wherein the target polymer film properties comprise a machine direction secant modulus, a transverse direction secant modulus, a dart impact strength, a haze total, a machine direction tear, a transverse direction tear, a machine direction tensile strength, a transverse direction tensile strength, a puncture break force, a puncture break energy, or any combination thereof.

Embodiment 18. The method of Embodiment 17, wherein the target polymer film properties comprise the machine direction secant modulus, the transverse direction secant modulus, the dart impact strength, the haze total, the machine direction tear, the transverse direction tear, the machine direction tensile strength, the transverse direction tensile strength, the puncture break force, and the puncture break energy.

Embodiment 19. The method of Embodiments 12-18, wherein the algorithm defines: the target polymer film properties as a function of a plurality of polymer properties; and at least a portion of the plurality of the polymer properties as a function of the reactor parameters.

Embodiment 20. The method of Embodiments 12-19, wherein the Gaussian process model-derived algorithm comprises a kernel function of K(xᵢ, xⱼ)=σ² exp(−(xᵢ−xⱼ)ⁿ/2L²)+δᵢⱼσ²ₙₒᵢₛₑ, where xᵢ is a vector of prediction variables at experiment i, xⱼ is a vector of the prediction variables at experiment j, σ and L are model hyper-parameters, δᵢⱼ is equal to zero when i and j and equal to one when i and j are equivalent, σ²ₙₒᵢₛₑ is a positive value, and n is a value ranging from one to five, inclusive.

Embodiment 21. A computing device for controlling a polymer production system, the computing device comprising: a processor; a memory coupled to the processor; and instructions provided to the memory, wherein the instructions are executable by the processor to perform the method of Embodiments 12-20.

Embodiment 22. A method for producing a polymer, the method comprising: generating polymer component parameters using a model comprising an algorithm with an input of target polymer properties of the polymer, wherein the target polymer properties comprise a density of the polymer, a melt index of the polymer, and a melt index ratio of the polymer, wherein the polymer comprises a plurality of polymer molecules, wherein the polymer component parameters comprise respective amounts of respective polymer molecules of the plurality of polymer molecules; and producing the polymer using the polymer component parameters.

Embodiment 23. The method of Embodiment 22, wherein the polymer comprises a copolymer of ethylene and hexene.

Embodiment 24. The method of Embodiment 22 or 23, wherein the polymer component parameters comprise respective amounts of methylene units of the respective polymer molecules.

Embodiment 25. The method of Embodiments 22-24, wherein the polymer component parameters comprise: respective amounts of long chain branches of the respective polymer molecules; and respective amounts of hexene-derived units of the respective polymer molecules.

Embodiment 26. The method of Embodiment 22-25, wherein generating the polymer component parameters comprises determining the density of the polymer based on a plurality of densities of the plurality of polymer molecules.

Embodiment 27. The method of Embodiment 26, wherein the plurality of densities are determined using an equation of

$${ρ}_{i} = {ρ}_{min} + \frac{( {ρ}_{max} - {ρ}_{min} )}{1 + exp ( - k * ( {log}_{10} n {CH}_{2} ) )}$$

where ρᵢ is a respective density of a respective polymer component i, ρₘᵢₙ is a minimum density of the respective polymer component i, ρₘₐₓ is a maximum density of the respective polymer component i, nCH₂ is a respective amount of methylene units in the respective polymer component i, and k is a value defined based on a number of butyl branches of the respective polymer component i.

Embodiment 28. The method of Embodiment 27, wherein:

$$k = \frac{α}{{{log}_{10} ( nButyl + 2 )}^{β}}$$

where α and β are model parameters, and nButyl is the number of butyl branches of the respective polymer component i.

Embodiment 29. The method of Embodiment 26 or 27, wherein ρₘₐₓ=c1−c2 log₁₀ MW, wherein c1 and c2 are model parameters, wherein MW is a molecular weight of the respective polymer component i.

Embodiment 30. The method of Embodiment 22-29, wherein generating the polymer component parameters comprises determining the melt index of the polymer based on a plurality of melt indexes of the plurality of polymer molecules.

Embodiment 31. The method of Embodiment 30, wherein the plurality of melt indexes are determined using an equation of

$$ln ( {MI}_{i} ) = a - b * ( {MW}_{backbone} + {MW}_{branch} ) + c * {MW}_{backbone} * {MW}_{branch}$$

where MIᵢ is a melt index of a respective polymer component i, MW_{backbone} is a backbone molecular weight of the respective polymer component i, MW_{branch} is a branch molecular weight of the respective polymer component i, and a, b, and c are model parameters.

Embodiment 32. A computing device for controlling a polymer production system, the computing device comprising: a processor; a memory coupled to the processor; and instructions provided to the memory, wherein the instructions are executable by the processor to perform the method of Embodiments 22-31.

Embodiment 33. A method for producing a polymer, the method comprising: generating polymer component parameters using a model comprising an algorithm with an input of target polymer properties of a polymer film, wherein the polymer film comprises the polymer, wherein the polymer comprises a plurality of polymer molecules, wherein the polymer component parameters comprise respective amounts of respective polymer molecules of the plurality of polymer molecules; and generating the polymer using the polymer component parameters.

Embodiment 34. The method of Embodiment 33, wherein the polymer comprises a copolymer of ethylene and hexene.

Embodiment 35. The method of Embodiment 33 or 34, wherein the target polymer film properties comprise a machine direction secant modulus, a transverse direction secant modulus, a dart impact strength, a haze total, a machine direction tear, a transverse direction tear, a machine direction tensile strength, a transverse direction tensile strength, a puncture break force, a puncture break energy, or any combination thereof.

Embodiment 36. The method of Embodiment 35, wherein the target polymer film properties comprise at least three of the machine direction secant modulus, the transverse direction secant modulus, the dart impact strength, the haze total, the machine direction tear, the transverse direction tear, the machine direction tensile strength, the transverse direction tensile strength, the puncture break force, or the puncture break energy.

Embodiment 37. The method of Embodiments 33-35, wherein the polymer component parameters comprise respective amounts of methylene units of the respective polymer molecules.

Embodiment 38. The method of Embodiments 33-37, wherein the polymer component parameters comprise: respective amounts of long chain branches of the respective polymer molecules; and respective amounts of hexene-derived units of the respective polymer molecules.

Embodiment 39. The method of Embodiments 33-38, comprising: generating one or more polymer properties based on the target polymer film properties; and producing the polymer component parameters based on the one or more polymer properties.

Embodiment 40. The method of Embodiment 39, wherein the one or more polymer properties comprise a density, a melt index, a melt index ratio, or any combination thereof.

Embodiment 41. The method of Embodiments 33-40, wherein producing the polymer comprises producing the polymer using at least one metallocene catalyst.

Embodiment 42. A computing device for controlling a polymer production system, the computing device comprising: a processor; a memory coupled to the processor; and instructions provided to the memory, wherein the instructions are executable by the processor to perform the method of Embodiments 33-42.

Embodiment 43. A method for producing a polymer, the method comprising: generating polymer properties of the polymer using a model comprising an algorithm with an input of a cross-fractionation characterization (CFC) of the polymer, wherein the CFC is generated based on a user input regarding one or more portions of the CFC; and producing the polymer having the polymer properties.

Embodiment 44. The method of Embodiment 43, wherein the one or more portions of the CFC correspond to one or more peaks of the CFC.

Embodiment 45. The method of Embodiment 44, where the one or more peaks comprise a first peak and a second peak respectively corresponding to a first component and a second component of the polymer, wherein the user input comprises a first weight ratio of the first component, a second weight ratio of the second component, a first molecular weight of the first component, and a second molecular weight of the second component.

Embodiment 46. The method of Embodiments 43-45, wherein the user input comprises a first CFC elution temperature of the first component and a second CFC elution temperature of the second component.

Embodiment 47. The method of Embodiments 43-46, wherein the polymer properties comprise a density, a melt index, a melt index ratio, or any combination thereof.

Embodiment 48. The method of Embodiments 43-47, wherein: the method comprises generating reactor parameters using a second model comprising a Gaussian process model-derived algorithm with an input of the polymer properties of the polymer; and producing the polymer comprises producing the polymer using the reactor parameters.

Embodiment 49. The method of Embodiment 48, wherein: the reactor parameters comprise at least two of: a reactor bed temperature, a hexene to ethylene feed flow ratio, a hydrogen to ethylene gas ratio, a reactor residence time, a partial pressure of ethylene, or an isopentane composition.

Embodiment 50. The method of Embodiments 43-49, wherein: the polymer comprises a plurality of polymer molecules; the method comprises determining polymer component parameters of the polymer; the polymer component parameters comprise respective amounts of respective polymer molecules of the plurality of polymer molecules; and generating the polymer properties comprises generating the polymer properties based on the polymer component parameters.

Embodiment 51. The method of Embodiments 43-50, wherein: the method comprises generating kinetic parameters based on the CFC; and producing the polymer comprises producing the polymer using the kinetic parameters.

Embodiment 52. The method of Embodiments 43-51, wherein the polymer comprises a linear low-density polyethylene (LLDPE).

Embodiment 53. A computing device for controlling a polymer production system, the computing device comprising: a processor, a memory coupled to the processor; and instructions provided to the memory, wherein the instructions are executable by the processor to perform the method of Embodiments 43-52.

Embodiment 54. A method for producing a polymer, the method comprising: generating polymer film properties of a polymer film using a model comprising an algorithm with an input of a cross-fractionation characterization (CFC) of the polymer, wherein the polymer film comprises the polymer, wherein the CFC is generated based on a user input regarding one or more portions of the CFC; and producing the polymer.

Embodiment 55. The method of Embodiment 54, wherein the polymer film properties comprise a machine direction secant modulus, a transverse direction secant modulus, a dart impact strength, a haze total, a machine direction tear, a transverse direction tear, a machine direction tensile strength, a transverse direction tensile strength, a puncture break force, a puncture break energy, or any combination thereof.

Embodiment 56. The method of Embodiment 55, wherein the polymer film properties comprise at least three of the machine direction secant modulus, the transverse direction secant modulus, the dart impact strength, the haze total, the machine direction tear, the transverse direction tear, the machine direction tensile strength, the transverse direction tensile strength, the puncture break force, or the puncture break energy.

Embodiment 57. The method of Embodiments 54-56, wherein: the one or more portions of the CFC correspond to one or more peaks of the CFC; and the user input defines how many peaks are included in the one or more peaks.

Embodiment 58. The method of Embodiments 54-57, comprising generating, using the model and based on the CFC, polymer properties of the polymer, wherein the polymer properties comprise a density, a melt index, and a melt index ratio.

Embodiment 59. The method of Embodiments 54-58, wherein: the polymer comprises a plurality of polymer molecules; the method comprises determining polymer component parameters of the polymer; the polymer component parameters comprise respective amounts of respective polymer molecules of the plurality of polymer molecules; and generating the polymer film properties comprises generating the polymer film properties based on the polymer component parameters.

Embodiment 60. The method of Embodiments 54-59, comprising generating reactor parameters based on the polymer film properties of the polymer; and producing the polymer using the reactor parameters.

Embodiment 61. The method of Embodiment 60, wherein the reactor parameters comprise: a catalyst composition having at least one metallocene catalyst; and at least two of: a reactor bed temperature, a hexene to ethylene feed flow ratio, a hydrogen to ethylene gas ratio, a reactor residence time, a partial pressure of ethylene, or an isopentane composition.

Embodiment 62. The method of Embodiments 54-61, wherein the polymer comprises a metallocene linear low-density polyethylene (mLLDPE).

Embodiment 63. A computing device for controlling a polymer production system, the computing device comprising: a processor; a memory coupled to the processor, and instructions provided to the memory, wherein the instructions are executable by the processor to perform the method of Embodiments 54-62.

1. A method for producing a polymer, the method comprising:
generating polymer properties of the polymer using a model comprising an algorithm with an input of a cross-fractionation characterization (CFC) of the polymer, wherein the CFC is generated based on a user input regarding one or more portions of the CFC; and
producing the polymer having the polymer properties.

2. The method of claim 1, wherein the one or more portions of the CFC correspond to one or more peaks of the CFC.

3. The method of claim 2, where the one or more peaks comprise a first peak and a second peak respectively corresponding to a first component and a second component of the polymer, wherein the user input comprises a first weight ratio of the first component, a second weight ratio of the second component, a first molecular weight of the first component, and a second molecular weight of the second component.

4. The method of claim 3, wherein the user input comprises a first CFC elution temperature of the first component and a second CFC elution temperature of the second component.

5. The method of claim 1, wherein the polymer properties comprise a density, a melt index, a melt index ratio, or any combination thereof.

6. The method of claim 1, wherein:
the method further comprises generating reactor parameters using a second model comprising a Gaussian process model-derived algorithm with an input of the polymer properties of the polymer; and
producing the polymer comprises producing the polymer using the reactor parameters.

7. The method of claim 6, wherein:
the reactor parameters comprise at least two of: a reactor temperature, a comonomer to monomer feed flow ratio, a hydrogen to monomer gas ratio, a reactor residence time, a partial pressure of monomer, and an induced condensing agent (ICA) composition.

8. The method of claim 6, wherein:
the polymer comprises a plurality of polymer molecules;
the method comprises determining polymer component parameters of the polymer;
the polymer component parameters comprise respective amounts of respective polymer molecules of the plurality of polymer molecules; and
generating the polymer properties comprises generating the polymer properties based on the polymer component parameters.

9. The method of claim 6, wherein:
the method comprises generating kinetic parameters based on the CFC; and
producing the polymer comprises producing the polymer using the kinetic parameters.

10. The method of claim 1, wherein the polymer comprises a linear low-density polyethylene (LLDPE).

11. A computing device for controlling a polymer production system, the computing device comprising:
a processor;
a memory coupled to the processor; and
instructions provided to the memory, wherein the instructions are executable by the processor to perform the method of claim 1.

12. A method for producing a polymer, the method comprising:
generating polymer film properties of a polymer film using a model comprising an algorithm with an input of a cross-fractionation characterization (CFC) of the polymer, wherein the polymer film comprises the polymer, wherein the CFC is generated based on a user input regarding one or more portions of the CFC; and
producing the polymer.

13. The method of claim 12, wherein the polymer film properties comprise a machine direction secant modulus, a transverse direction secant modulus, a dart impact strength, a haze total, a machine direction tear, a transverse direction tear, a machine direction tensile strength, a transverse direction tensile strength, a puncture break force, a puncture break energy, or any combination thereof.

14. The method of claim 13, wherein the polymer film properties comprise at least three of the machine direction secant modulus, the transverse direction secant modulus, the dart impact strength, the haze total, the machine direction tear, the transverse direction tear, the machine direction tensile strength, the transverse direction tensile strength, the puncture break force, and the puncture break energy.

15. The method of claim 12, wherein:
the one or more portions of the CFC correspond to one or more peaks of the CFC; and
the user input defines how many peaks are included in the one or more peaks.

16. The method of claim 12, comprising generating, using the model and based on the CFC, polymer properties of the polymer, wherein the polymer properties comprise a density, a melt index, and a melt index ratio.

17. The method of claim 12, wherein:
the polymer comprises a plurality of polymer molecules;
the method comprises determining polymer component parameters of the polymer;
the polymer component parameters comprise respective amounts of respective polymer molecules of the plurality of polymer molecules; and
generating the polymer film properties comprises generating the polymer film properties based on the polymer component parameters.

18. The method of claim 17, comprising
generating reactor parameters based on the polymer film properties of the polymer; and
producing the polymer using the reactor parameters.

19. The method of claim 18 wherein the reactor parameters comprise:
a catalyst composition having at least one metallocene catalyst; and
at least two of: a reactor bed temperature, a hexene to ethylene flow ratio, a hydrogen to ethylene gas ratio, a reactor residence time, a partial pressure of ethylene, or an isopentane composition.

20. The method of claim 12, wherein the polymer comprises a metallocene linear low-density polyethylene (mLLDPE).
